(19) United States
(12) Patent Application Publication
(10) Pub. No.: US 2025/0205073 A1
(43) Pub. Date: Jun. 26, 2025

(54) Attachment System for Orthopedic Supports

(71) Applicant: Cast21, Inc., Chicago, IL (US)

(72) Inventors: Ashley S. Moy, Chicago, IL (US); Veronica A. Hogg-Cornejo, Chicago, IL (US); Abhilash Seshadri, Chicago, IL (US); Christian Guerrero, Chicago, IL (US); Yaser Kazmi, Chicago, IL (US); Lindsay Kwok, Chicago, IL (US); Jason Troutner, Chicago, IL (US)

(21) Appl. No.: 18/850,908

(22) PCT Filed: Mar. 28, 2023
(86) PCT No.: PCT/US2023/016628

§ 371 Date: Sep. 25, 2024

Related U.S. Application Data
(60) Provisional application No. 63/324,321, filed on Mar. 28, 2022.
(60) Provisional application No. 63/411,697, filed on Sep. 30, 2022.

(51) Int. Cl. A61F 5/058; A61F 5/01

(52) U.S. Cl. CPC A61F 5/05875; A61F 5/0118

(57) ABSTRACT

The present disclosure provides attachment systems to a a parent device, namely, a latticed orthopedic support, and accessory devices for a variety of achievements.

### CROSS-REFERENCE TO RELATED APPLICATIONS

This application claims priority to (i) U.S. Provisional Patent Application No. 63/324,321, filed Mar. 28, 2022, and (ii) U.S. Provisional Patent Application No. 63/411,697, filed Sep. 30, 2022, the contents of each of which are hereby incorporated by reference in their entirety.

### TECHNICAL FIELD

The present disclosure relates to orthotics and orthopedic supports such as casts, braces, and splints; specifically, for the immobilization of one or more joints and limbs.

### BACKGROUND

Unless otherwise indicated herein, the materials described in this section are not prior art to the claims in this application and are not admitted to be prior art by inclusion in this section.

Millions of individuals experience bone fractures each year. Yet, non-surgical interventions for the treatment of broken bones have progressed little in the way of innovation. Some companies have sought to produce pre-formed or pre-fabricated braces to overcome some of the shortcomings of traditional casts. Some braces can immobilize multiple hand and wrist joints; however, they are a single, non-ambidextrous, form factor. These prefabricated products are limited in moldability, which is necessary for proper healing of fractures and can increase stock-keeping unit (SKU) inventory by six times when compared to the number of fiberglass or plaster products a clinic must hold for wrist and hand joint immobilization alone.

Further, these pre-fabricated products can negatively impact the patient's dexterity when applied to the hand or wrist, as there are limited options for customizing fit to each person's unique body shape. Additionally, all standards of care for casts and braces trap heat, sweat, and moisture against the patient's skin. Accordingly, there is a need for improved orthopedic supports.

### SUMMARY

The present disclosure is related to orthotics and orthopedics supports, such as casts, braces, and splints. Specifically, the present disclosure describes methods used to immobilize at least one joint with modular components. Further, it describes the methods by which objects may be attached in a standardized manner for therapeutic purposes. In particular, the present disclosure provides multiple modular components which are assembled to create an orthopedic support. This is the first time standardized, interchangeable parts for the immobilization of multiple joints for recovery, therapy, and personalization have been introduced.

The system described herein takes advantage of orthopedic support systems that contain apertures and are rigid. Preferred methods include fillable networks of channels that receive a liquid resin before hardening into a rigid structure. Such a system is described in U.S. Pat. No. 11,266,761, entitled “System for Forming a Rigid Support”, the contents of which is incorporated by reference herein in their entirety.

The latticed orthopedic support shown in U.S. Pat. No. 11,266,761 contains edges and intersections of support material as well as areas void of support material. This unique design can serve as a platform for modular attachments to accomplish numerous goals such as additional immobilization of additional joints, gripping tools, and patient personalization.

The present disclosure describes attachment systems to a parent device, namely, the orthopedic support, and accessory devices for a variety of achievements. Because of the complex structure of the latticed brace solutions, there are not any existing methods or inventions that are specifically useful for attaching accessories or creating other opportunities for personalization on the orthopedic support. This circumstance led the inventors to invent the following set of solutions to allow a user to immobilize more than one joint, hold utensils, or customize or add to the appearance of their device.

Each attachment system includes (a) a parent device that immobilizes a body part of the patient and includes apertures in its final form and (b) an accessory device that securely attaches to the rigid parent device. The purpose of the accessory device depends on the embodiment, and the specific attachment systems can be interchanged with their accessories. Although the figures and description provided herein mainly discusses immobilizing the wrist and arm of a patient, the parent device may be used to immobilize any body part.

While the system inventions are susceptible to embodiments of varying forms, shown are drawings of the preferred embodiments of the system inventions and methods for using the same will be detailed herein. However, it should be understood that the present disclosure is an exemplification of the principles of the invention and not intended to limit the spirit or scope of the system and/or the claims of the embodiments illustrated.

In particular, in a first aspect, the present disclosure provides device configured to be coupled to a lattice structure, wherein the lattice structure is configured to support a first joint of a body, and wherein the lattice structure defines a plurality of apertures configured to allow for a flow of air and water to the first joint. The device includes a strut having a first end and a second end opposite the first end. The device also includes a coupling mechanism coupled to the first end of the strut, wherein the coupling mechanism is configured to couple the device to the lattice structure. The device also includes an immobilization component coupled to the strut, wherein the immobilization component is configured to support a second joint of the body to thereby at least partially immobilize the second joint.

In a second aspect, the present disclosure provides device configured to be coupled to a lattice structure, wherein the lattice structure is configured to support a first joint of a body, and wherein the lattice structure defines a plurality of apertures configured to allow for a flow of air and water to the first joint. The device includes a strap having a first end and a second end opposite the first end. The device also includes a coupling mechanism coupled to the first end of the strap, wherein the coupling mechanism is configured to couple the device to the lattice structure. The device also includes an immobilization component configured to be held in place by the strap, wherein the immobilization component is configured to support a second joint of the body to thereby at least partially immobilize the second joint.

In a third aspect, the present disclosure provides device configured to be coupled to a lattice structure, wherein the lattice structure is configured to support a first joint of a body, and wherein the lattice structure defines a plurality of apertures configured to allow for a flow of air and water to the first joint. The device includes a first elastic cable having a first end and a second end opposite the first end. The device also includes a first coupling mechanism coupled to the first end of the first elastic cable, wherein the first coupling mechanism is configured to couple the first elastic cable to a first aperture of the plurality of apertures of the lattice structure. The second end of the first elastic cable and the second end of the second elastic cable are configured to hold a utensil.

In a fourth aspect, the present disclosure provides device configured to be coupled to a lattice structure, wherein the lattice structure is configured to support a first joint of a body, and wherein the lattice structure defines a plurality of apertures configured to allow for a flow of air and water to the first joint. The device includes a first elastic cable having a first end and a second end opposite the first end. The device also includes a first coupling mechanism positioned at the first end of the first elastic cable, wherein the first coupling mechanism is configured to couple the first elastic cable to an aperture of the plurality of apertures of the lattice structure. The device also includes a second coupling mechanism positioned at the second end of the first elastic cable, wherein the second coupling mechanism is configured to couple the first elastic cable to a first digit of the body. The device also includes a second elastic cable having a first end and a second end opposite the first end. The device also includes a third coupling mechanism positioned at the first end of the second elastic cable, wherein the third coupling mechanism is configured to couple the second elastic cable to an aperture of the plurality of apertures of the lattice structure. The device also includes a fourth coupling mechanism positioned at the second end of the second elastic cable, wherein the fourth coupling mechanism is configured to couple the second elastic cable to a second digit of the body.

In a fifth aspect, the present disclosure provides device configured to be coupled to a lattice structure, wherein the lattice structure is configured to support a first joint of a body, and wherein the lattice structure defines a plurality of apertures configured to allow for a flow of air and water to the first joint. The device includes a through-hole positioned at a first end of the device, wherein the through-hole is configured to receive at least a portion of a thumb of the body when in use. The device also includes a utensil positioned at a second end of the device opposite the first end. The device also includes an attachment mechanism configured to be removably positioned at least partially within an aperture of the plurality of apertures of the lattice structure.

In a sixth aspect, the present disclosure provides a method of forming a support for application to a body area. The method includes (a) positioning a lattice structure around the body area, (b) coupling a first portion of the lattice structure to a second portion of the lattice structure via a coupling mechanism, and (c) injecting a liquid resin and a catalyst mixture into a lumen of the lattice structure, wherein the liquid resin and the catalyst mixture transforms into a solid in the lumen of the lattice structure such that the lattice structure hardens into the support for application to the body area.

In a seventh aspect, the present disclosure provides a method of forming a support for application to a body area. The method includes (a) positioning a lattice structure around the body area, (b) coupling a restraining clip to a portion of the lattice structure, and (c) injecting a liquid resin and a catalyst mixture into a lumen of the lattice structure, wherein the liquid resin and the catalyst mixture transforms into a solid in the lumen of the lattice structure such that the lattice structure hardens into the support for application to the body area, and wherein the restraining clip prevents the liquid resin and the catalyst mixture from entering a portion of the lumen thereby creating a void in the lumen.

These, as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a device for supporting a joint of a body, according to an example embodiment.

FIG. 2A illustrates an example coupling mechanism of the device of FIG. 1, according to an example embodiment.

FIG. 2B illustrates another example coupling mechanism of the device of FIG. 1, according to an example embodiment.

FIG. 3 illustrates another device for supporting a joint of a body, according to an example embodiment.

FIG. 4 illustrates another device for supporting a joint of a body, according to an example embodiment.

FIG. 5 illustrates an example strut of one of the devices of FIGS. 1-4, according to an example embodiment.

FIG. 6 illustrates an example immobilization component of one of the devices of FIGS. 1-4, according to an example embodiment.

FIG. 7 illustrates an example coupling mechanism of one of the devices of FIGS. 1-4, according to an example embodiment.

FIG. 8 illustrates the example coupling mechanism of FIG. 7, according to an example embodiment.

FIG. 9 illustrates a foam pad for use with one of the devices of FIGS. 1-4, according to an example embodiment.

FIG. 10 illustrates another device for supporting a joint of a body, according to an example embodiment.

FIG. 11 illustrates another device for supporting a joint of a body, according to an example embodiment.

FIG. 12 illustrates a device for securing a utensil, according to an example embodiment.

FIG. 13 illustrates another device for securing a utensil, according to an example embodiment.

FIG. 14 illustrates another device for securing a utensil, according to an example embodiment.

FIG. 15 illustrates a rehabilitation device for use with a lattice support structure, according to an example embodiment.

FIG. 16 illustrates a utensil for use with a lattice support structure, according to an example embodiment.

FIG. 17 illustrates an example system for connecting lattice support structures, according to an example embodiment.

FIG. 18A illustrates another example system for connecting lattice support structures, according to an example embodiment.

FIG. 18B illustrates another example system for connecting lattice support structures, according to an example embodiment.

FIG. 19 illustrates another example system for connecting lattice support structures, according to an example embodiment.

FIG. 20 illustrates another example system for connecting lattice support structures, according to an example embodiment.

FIG. 21 illustrates an example coupling mechanism for connecting lattice support structures, according to an example embodiment.

FIG. 22 illustrates another example system for connecting lattice support structures, according to an example embodiment.

FIG. 23 illustrates another example system for connecting lattice support structures, according to an example embodiment.

FIG. 24 illustrates another example system for connecting lattice support structures, according to an example embodiment.

FIG. 25 illustrates an example lattice support structure, according to an example embodiment.

FIG. 26 illustrates another example system for connecting lattice support structures, according to an example embodiment.

FIG. 27 illustrates another example system for connecting lattice support structures, according to an example embodiment.

FIG. 28 illustrates another example system for connecting lattice support structures, according to an example embodiment.

FIG. 29 illustrates another example system for connecting lattice support structures, according to an example embodiment.

FIG. 30 illustrates another example system for connecting lattice support structures, according to an example embodiment.

FIG. 31 illustrates another example system for connecting lattice support structures, according to an example embodiment.

FIG. 32A illustrates an example system forming a support for application to a body area, according to an example embodiment.

FIG. 32B illustrates an example system forming a support for application to a body area, according to an example embodiment.

FIG. 33 illustrates another example system forming a support for application to a body area, according to an example embodiment.

FIG. 34 illustrates another example system forming a support for application to a body area, according to an example embodiment.

FIG. 35 illustrates another example system forming a support for application to a body area, according to an example embodiment.

FIG. 36 illustrates another example system forming a support for application to a body area, according to an example embodiment.

FIG. 37 illustrates another example system forming a support for application to a body area, according to an example embodiment.

FIG. 38 illustrates another example system forming a support for application to a body area, according to an example embodiment.

FIG. 39 illustrates an example dispensing pin for supplying a mixture to the lattice structure, according to an example embodiment.

FIG. 40 illustrates an example cylindrical casing including a piston used to push a mixture to the dispensing pin of FIG. 39.

FIG. 41 illustrates a pressure point and a hemi-spherical foam padding configured to be used with the lattice support structure, according to an example embodiment.

### DETAILED DESCRIPTION

Example methods and systems are described herein. It should be understood that the words “example,” “exemplary,” and “illustrative” are used herein to mean “serving as an example,” “instance,” or “illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Reference to, e.g., a “first” item does not require or preclude the existence of, e.g., a “second” or higher-numbered item. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

As used herein, the term “orthopedic support” generally refers to orthotics and orthopedic casts, braces, and splits, specifically for the immobilization of one or more joints or limbs.

As used herein, the term “component” generally refers to a singular unit of a modular support system, specifically for the orthopedic support and immobilization of a localized limb or joint.

As used herein, the term “parent device” generally refers to a singular component in a modular support system, specifically the initial device placed on the body within the system for support and immobilization of a localized limb or joint.

As used herein, the term “accessory” generally refers to a singular component in a modular support system, specifically the device in a system placed in tandem with the parent device, for the support and immobilization of a limb or joint adjacent to the limb or joint being immobilized by the parent device.

As used herein, the term “adornment” generally refers to an external object, device, or product secured onto a component to create a personalized support apparatus.

Generally, the present disclosure provides attachment mechanisms of an accessory to a parent device, where the parent device comprises a lattice structure that immobilizes a single joint or body area. These embodiments describe specific attachment systems for numerous accessories; however, it should be noted that these combinations can be executed interchangeably unless otherwise noted.

Thus, according to an embodiment, the present disclosure provides a device configured to be coupled to a lattice structure, wherein the lattice structure is configured to support a first joint of a body, and wherein the lattice structure defines a plurality of apertures configured to allow for a flow of air and water to the first joint. The device includes a strut having a first end and a second end opposite the first end. The device also includes a coupling mechanism coupled to the first end of the strut, wherein the coupling mechanism is configured to couple the device to the lattice structure. The device also includes an immobilization component coupled to the strut, wherein the immobilization component is configured to support a second joint of the body to thereby at least partially immobilize the second joint.

In an embodiment, the immobilization component is coupled to the second end of the strut.

In an embodiment, the strut is rigid.

In an embodiment, a length of the strut is adjustable.

In an embodiment, the immobilization component comprises a semi-circular shape.

In an embodiment, the immobilization component comprises a circular shape.

In an embodiment, the immobilization component includes a closed-cell foam or a silicone material configured to contact the second joint of the body.

In an embodiment, the coupling mechanism is configured to be removable coupled to the lattice structure.

In an embodiment, the coupling mechanism comprises a flexible open ring structure configured to transition from an open position in which the coupling mechanism is positioned around a portion of the lattice structure and a closed position in which the coupling mechanism secures the device to the lattice structure.

In an embodiment, wherein the coupling mechanism is configured to be fixed to the lattice structure.

In an embodiment, the coupling mechanism comprises a rigid open ring structure with a fixed opening, wherein the lattice structure is configured to receive a liquid resin and a catalyst mixture that transforms into a solid such that the lattice structure hardens to thereby support the first joint of the body, wherein the rigid open ring structure is configured to be positioned around the lattice structure prior to the lattice structure receiving the liquid resin and the catalyst mixture, and wherein a diameter of the lattice structure is larger than the fixed opening after receiving the liquid resin and the catalyst mixture such that the coupling mechanism is permanently fixed to the lattice structure once the liquid resin and the catalyst mixture transforms into a solid.

In an embodiment, the coupling mechanism a first coupling mechanism configured to be coupled to a first aperture of the plurality of apertures of the lattice structure, and a second coupling mechanism configured to be coupled to a second aperture of the plurality of apertures of the lattice structure.

In an embodiment, the coupling mechanism comprises a first coupling mechanism configured to be coupled to a first aperture of the plurality of apertures of the lattice structure, and a second coupling mechanism configured to be coupled to the first aperture of the plurality of apertures of the lattice structure.

In an embodiment, the strut comprises a first strut, the device further comprising a second strut having a first end and a second end, wherein the coupling mechanism coupled to the first end of the second strut, and wherein the immobilization component coupled to the second strut.

In an embodiment, the immobilization component comprises a first closed ring positioned adjacent the second end of the strut, and a second closed ring positioned between the first closed ring and the first end of the strut.

In an embodiment, a diameter of the immobilization component is adjustable.

In an embodiment, the strut includes a first through-hole including a first plurality of teeth, and a second through-hole including a second plurality of teeth, wherein the first plurality of teeth and the second plurality of teeth are configured to hold the immobilization component in place.

In another embodiment, the present disclosure provides a device configured to be coupled to a lattice structure, wherein the lattice structure is configured to support a first joint of a body, and wherein the lattice structure defines a plurality of apertures configured to allow for a flow of air and water to the first joint. The device includes a strap having a first end and a second end opposite the first end. The device also includes a coupling mechanism coupled to the first end of the strap, wherein the coupling mechanism is configured to couple the device to the lattice structure. The device also includes an immobilization component configured to be held in place by the strap, wherein the immobilization component is configured to support a second joint of the body to thereby at least partially immobilize the second joint.

In an embodiment, at least a portion of the strap includes a hook and loop fastener.

In an embodiment, the strap is wrapped around the immobilization component and then attached to itself to thereby secure the immobilization component to the second joint.

In an embodiment, the immobilization component is rigid.

In an embodiment, the second end of the strap includes a second coupling mechanism, and wherein the second coupling mechanism is configured to couple the second end of the strap to the lattice structure.

In an embodiment, the coupling mechanism comprises one of a t-anchor, an s-hook, or a c-hook.

In another embodiment, the present disclosure provides a device configured to be coupled to a lattice structure, wherein the lattice structure is configured to support a first joint of a body, and wherein the lattice structure defines a plurality of apertures configured to allow for a flow of air and water to the first joint. The device includes a first elastic cable having a first end and a second end opposite the first end. The device also includes a first coupling mechanism coupled to the first end of the first elastic cable, wherein the first coupling mechanism is configured to couple the first elastic cable to a first aperture of the plurality of apertures of the lattice structure. The second end of the first elastic cable and the second end of the second elastic cable are configured to hold a utensil.

In an embodiment, the first coupling mechanism and the second coupling mechanism comprise one of a t-anchor, an s-hook, or a c-hook.

In an embodiment, the device further includes a second elastic cable having a first end and a second end opposite the first end. The device further includes a second coupling mechanism coupled to the first end of the second elastic cable, wherein the second coupling mechanism is configured to couple the second elastic cable to a second aperture of the plurality of apertures of the lattice structure. The device further includes a third coupling mechanism positioned at the second end of the first elastic cable. The device further includes a fourth coupling mechanism positioned at the second end of the second elastic cable. The third coupling mechanism and the fourth coupling mechanism are configured to hold the utensil.

In another embodiment, the present disclosure provides a device configured to be coupled to a lattice structure, wherein the lattice structure is configured to support a first joint of a body, and wherein the lattice structure defines a plurality of apertures configured to allow for a flow of air and water to the first joint. The device includes a first elastic cable having a first end and a second end opposite the first end. The device further includes a first coupling mechanism positioned at the first end of the first elastic cable, wherein the first coupling mechanism is configured to couple the first elastic cable to an aperture of the plurality of apertures of the lattice structure. The device further includes a second coupling mechanism positioned at the second end of the first elastic cable, wherein the second coupling mechanism is configured to couple the first elastic cable to a first digit of the body. The device further includes a second elastic cable having a first end and a second end opposite the first end. The device further includes a third coupling mechanism positioned at the first end of the second elastic cable, wherein the third coupling mechanism is configured to couple the second elastic cable to an aperture of the plurality of apertures of the lattice structure. The device further includes a fourth coupling mechanism positioned at the second end of the second elastic cable, wherein the fourth coupling mechanism is configured to couple the second elastic cable to a second digit of the body.

In an embodiment, The device further includes a third elastic cable having a first end and a second end opposite the first end. The device further includes a fifth coupling mechanism positioned at the first end of the third elastic cable, wherein the fifth coupling mechanism is configured to couple the third elastic cable to an aperture of the plurality of apertures of the lattice structure. The device further includes a sixth coupling mechanism positioned at the second end of the third elastic cable, wherein the sixth coupling mechanism is configured to couple the third elastic cable to a third digit of the body. The device further includes a fourth elastic cable having a first end and a second end opposite the first end. The device further includes a seventh coupling mechanism positioned at the first end of the fourth elastic cable, wherein the seventh coupling mechanism is configured to couple the fourth elastic cable to an aperture of the plurality of apertures of the lattice structure. The device further includes an eighth coupling mechanism positioned at the second end of the fourth elastic cable, wherein the eighth coupling mechanism is configured to couple the fourth elastic cable to a fourth digit of the body.

In an embodiment, the present disclosure provides a device configured to be coupled to a lattice structure, wherein the lattice structure is configured to support a first joint of a body, and wherein the lattice structure defines a plurality of apertures configured to allow for a flow of air and water to the first joint. The device includes a through-hole positioned at a first end of the device, wherein the through-hole is configured to receive at least a portion of a thumb of the body when in use. The device further includes a utensil positioned at a second end of the device opposite the first end. The device further includes an attachment mechanism configured to be removably positioned at least partially within an aperture of the plurality of apertures of the lattice structure.

In an embodiment, the attachment mechanism comprises a protrusion configured to contact an edge of the aperture to thereby prevent slipping or turning of the device when in use.

In an embodiment, the utensil positioned at the second end of the device comprises one of a spoon, a fork, or a knife.

In another embodiment, the present disclosure provides a system including a first lattice structure, wherein the first lattice structure is configured to support a first area of a body, and wherein the first lattice structure defines a first plurality of apertures configured to allow for a flow of air and water to the first area of the body. The system further includes a second lattice structure, wherein the second lattice structure is configured to support a second area of the body, and wherein the second lattice structure defines a second plurality of apertures configured to allow for a flow of air and water to the second area of the body. The system further includes a coupling mechanism configured to couple the first lattice structure to the second lattice structure.

In an embodiment, the coupling mechanism comprises a plurality of struts each having a first end and a second end opposite the first end, wherein the first end of each of the plurality of struts are coupled to the first plurality of apertures of the first lattice structure, and wherein the second end of each of the plurality of struts are coupled to the second plurality of apertures of the second lattice structure.

In an embodiment, the coupling mechanism comprises a pairing mechanism.

In an embodiment, the coupling mechanism comprises a male protrusion and a female protrusion to thereby establish a fluidic connection between the first lattice structure and the second lattice structure.

In an embodiment, the coupling mechanism comprises an adhesive.

In an embodiment, the system further includes a third lattice structure, wherein the third lattice structure is configured to support a third area of the body, and wherein the third lattice structure defines a third plurality of apertures configured to allow for a flow of air and water to the third area of the body, and a second coupling mechanism configured to couple the second lattice structure to the third lattice structure.

In an embodiment, the coupling mechanism is configured to adjust a distance between the first lattice structure and the second lattice structure.

In an embodiment, the system further includes a semi-rigid base coupled to the first lattice structure.

In an embodiment, an anterior section of the semi-rigid base comprises a foam material.

In another embodiment, the present disclosure provides a method of forming a support for application to a body area. The method includes (a) positioning a lattice structure around the body area, (b) coupling a first portion of the lattice structure to a second portion of the lattice structure via a coupling mechanism, and (c) injecting a liquid resin and a catalyst mixture into a lumen of the lattice structure, wherein the liquid resin and the catalyst mixture transforms into a solid in the lumen of the lattice structure such that the lattice structure hardens into the support for application to the body area.

In an embodiment, the coupling mechanism comprises a locking strap having a first protrusion configured to be positioned within a first aperture of the first portion of the lattice structure and a second protrusion configured to be positioned within a second aperture of the second portion of the lattice structure.

In an embodiment, the second portion of the lattice structure includes a through-hole, and wherein the first portion of the lattice structure includes an extension configured to be positioned through the through-hole, wherein a diameter of the extension is greater than a diameter of the through-hole after the liquid resin and the catalyst mixture transforms into a solid.

In an embodiment, the coupling mechanism comprises a plurality of hook and fastener straps.

In another embodiment, the present disclosure provides a method of forming a support for application to a body area. The method includes (a) positioning a lattice structure around the body area, (b) coupling a restraining clip to a portion of the lattice structure, and (c) injecting a liquid resin and a catalyst mixture into a lumen of the lattice structure, wherein the liquid resin and the catalyst mixture transforms into a solid in the lumen of the lattice structure such that the lattice structure hardens into the support for application to the body area, and wherein the restraining clip prevents the liquid resin and the catalyst mixture from entering a portion of the lumen thereby creating a void in the lumen.

In an embodiment, the void in the lumen creates a flexible section of the lattice structure.

### A. Immobilization

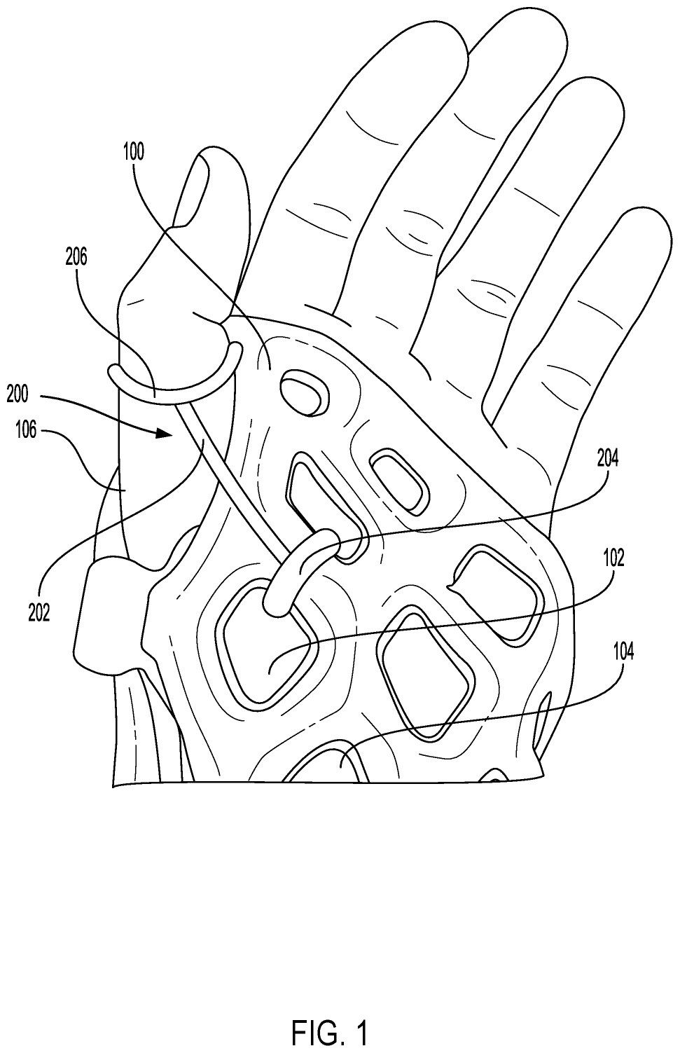

With reference to FIGS. 1-11, the present disclosure provides a device 200 configured to be coupled to a lattice structure 100. Throughout the specification, the lattice structure 100 may comprise any of the features disclosed in U.S. Pat. No. 11,266,761, which is hereby incorporated by reference in its entirety. As shown in FIG. 1, the lattice structure 100 is configured to support a first joint 104 of a body, and the lattice structure 100 defines a plurality of apertures 102 configured to allow for a flow of air and water to the first joint 104. As further shown in FIG. 1, the device 200 includes a strut 202 having a first end and a second end opposite the first end. The device 200 also includes a coupling mechanism 204 coupled to the first end of the strut. The coupling mechanism 204 is configured to couple the device 200 to the lattice structure 100. The device 200 further includes an immobilization component 206 coupled to the strut 202. The immobilization component 206 is configured to support a second joint 106 of the body to thereby at least partially immobilize the second joint 106.

In one example, the immobilization component 206 is coupled to the second end of the strut. In one example, the strut 202 is rigid. In another example, the strut 202 is flexible. In another example, a length of the strut 202 is adjustable. In one example, the immobilization component 206 comprises a semi-circular shape. In another example, the immobilization component 206 comprises a circular shape. In one example, the immobilization component includes a closed-cell foam or a silicone material configured to contact the second joint of the body, thereby providing comfort to the user.

In one example, the coupling mechanism 204 is configured to be removably coupled to the lattice structure 100. In one such example, the coupling mechanism 204 comprises a flexible open ring structure configured to transition from an open position in which the coupling mechanism 204 is positioned around a portion of the lattice structure 100 and a closed position in which the coupling mechanism 204 secures the device 200 to the lattice structure 100. As such, the opening of the flexible open ring structure is bigger in the open position, and then snaps back to the smaller closed position due to the material properties of the flexible open ring.

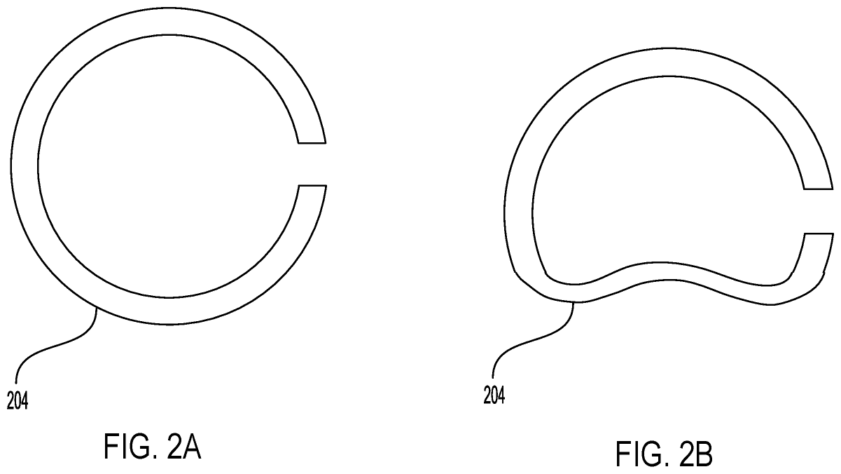

In one example, after the lattice structure 100 is in its rigid form the device 200 can be secured to the edges of an aperture 102 with a flexible, semi-rigid open-ring coupling mechanism 204 that slides onto the lattice structure 100. This open-ring structure can be manufactured through various methods, including injection molding of resins or silicones or 3D printing. In one example, as shown in FIG. 2A, the coupling mechanism 204 can be circular in shape to lay flush with the lattice structure 100. In another example, as shown in FIG. 2B, the coupling mechanism 204 can curve up into a kidney bean shape on the patient side of the device such that the coupling mechanism 204 does not contact the skin of the user when in use, thereby eliminating the possibility for the end of the coupling mechanism 204 to cause patient discomfort. In one embodiment, the strut 202 is stationary and with a predetermined length and angle of orientation. Further, in one embodiment, the strut 202 can be constructed out of a graphite fiber or other metal or resin such that the forces required to fracture the strut are lower than that of bone and that do not rust when exposed to moisture. The strut 202 can be fabricated already connected to the semi-rigid open-ring with a manufacturing method such as 3D printing or snapped into place after manufacturing both the coupling mechanism 204 and the strut 202; the strut 202 can be compressed into a socket or trench on the semi-rigid open-ring. In one example, the strut 202, the coupling mechanism 204, and the immobilization component 206 are a single molded unit. In another example, the strut 202 and the immobilization component 206 are a single molded unit, while the coupling mechanism 204 is removably coupled to the strut 202. In yet another example, the coupling mechanism 204, and the immobilization component 206 are each removably coupled to one another.

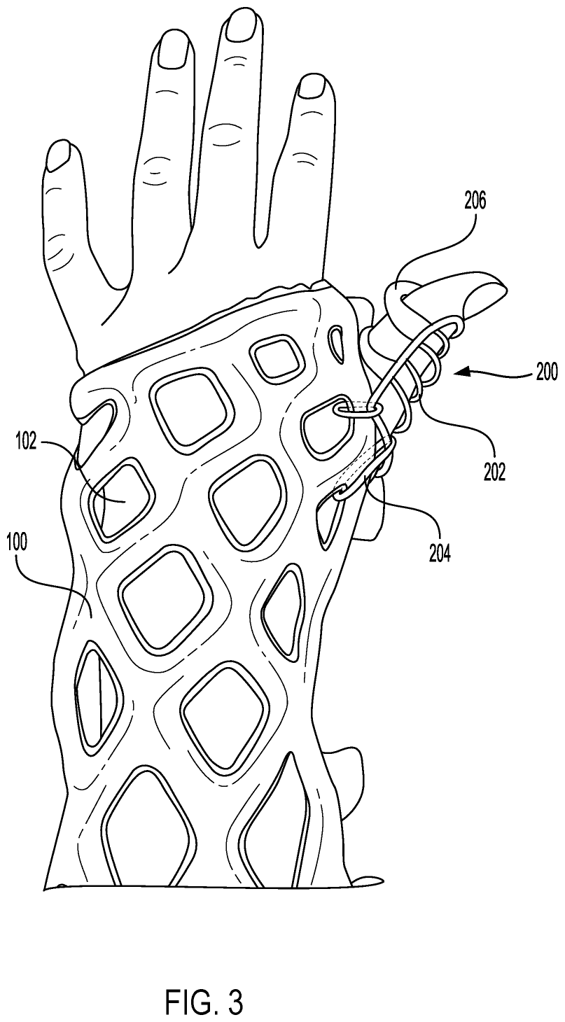

In one example, as shown in FIG. 3, the coupling mechanism 204 comprises a first coupling mechanism configured to be coupled to a first aperture of the plurality of apertures 102 of the lattice structure 100, and a second coupling mechanism configured to be coupled to a second aperture of the plurality of apertures 102 of the lattice structure 100. FIG. 3 further shows a variation of the immobilization component 206 and strut 202 around the body area, securing at least secondary joints and latching back to the strut 202. The immobilization component 206 can be fabricated with an elastic ring clip or strap, supported by a supporting strut 202 made with a flexible and moldable material such as a combination of plastics, aluminum, and foam.

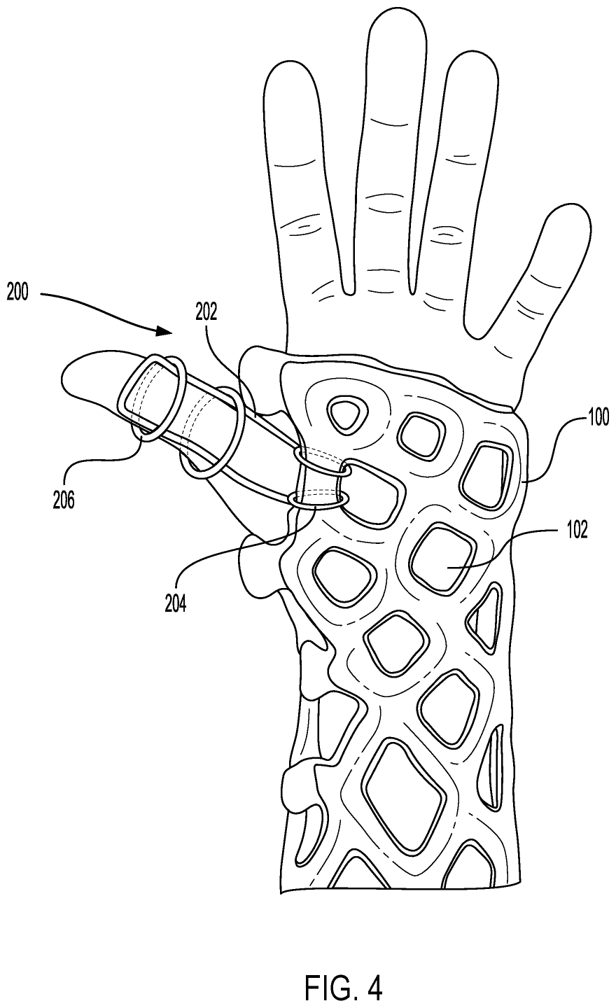

In another example, as shown in FIG. 4, the coupling mechanism 204 comprises a first coupling mechanism configured to be coupled to a first aperture of the plurality of apertures 102 of the lattice structure 100, and a second coupling mechanism configured to be coupled to the first aperture of the plurality of apertures 102 of the lattice structure 100. FIG. 4 further shows a variation of the immobilization component 206 and strut 202 as a combination whereas at least two struts are used to support on two or more sides. The supporting struts 202 are held in position by straps or a closed ring. The struts 202 may be secured to the lattice structure 100 through a C hook (the strut can be fabricated with flexible plastics or a moldable material stack up similar to the lattice structure 100). In one example, as shown in FIG. 4, the immobilization component 206 comprises a first closed ring positioned adjacent the second end of the strut 202 and a second closed ring positioned between the first closed ring and the first end of the strut 202. The ring can have material properties that will allow the joint to remain completely immobile or allow for partial movement.

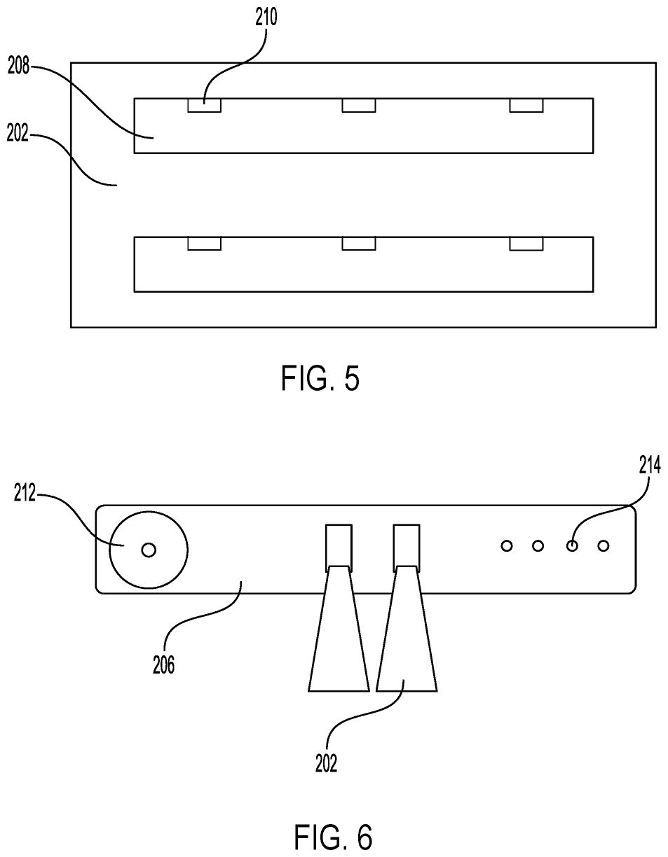

FIG. 5 shows a detailed view of a design variation the strut 202 of the device 200. The strut 202, made from a rigid material, consists of a railing mechanism 208 with teeth 210 that can be used to adjust sizing for the immobilization component 206 (such as the closed rings discussed above). In particular, as shown in FIG. 5, the strut 202 may comprise a first through-hole including a first plurality of teeth and a second through-hole including a second plurality of teeth, wherein the first plurality of teeth and the second plurality of teeth are configured to hold the immobilization component 206 in place.

In one example, a diameter of the immobilization component 206 is adjustable. In one such example, as shown in FIG. 6, the immobilization component 206 is a belt-like system made of a flexible material that can be wrapped around the body area for immobilizing at least one secondary joint. In one embodiment, a padding layer is deposited along at least a portion of the surface of the immobilization component 206 that contacts the body of the user. A rivet 212 and corresponding apertures 214 may be used to allow variations for sizing of the body area. The struts 202 can be secured to the end cap through the welding or pressure sensitive adhesives. In a design variation, pegs could be placed along the struts and the immobilization component 206 could rivet directly to the struts 202.

The device 200 described herein can be applied to one or more edges of the lattice structure 100 to secure one or more different joints. FIG. 1 demonstrates the semi-rigid open-ring attachment in plurality to secure a single joint, the thumb interphalangeal joint. Additional accessories can be attached to secure additional digits. If a flat or curved “cap” is used at the distal end of the strut 202, at least two accessories of this design may be used to immobilize the joint from different directions.

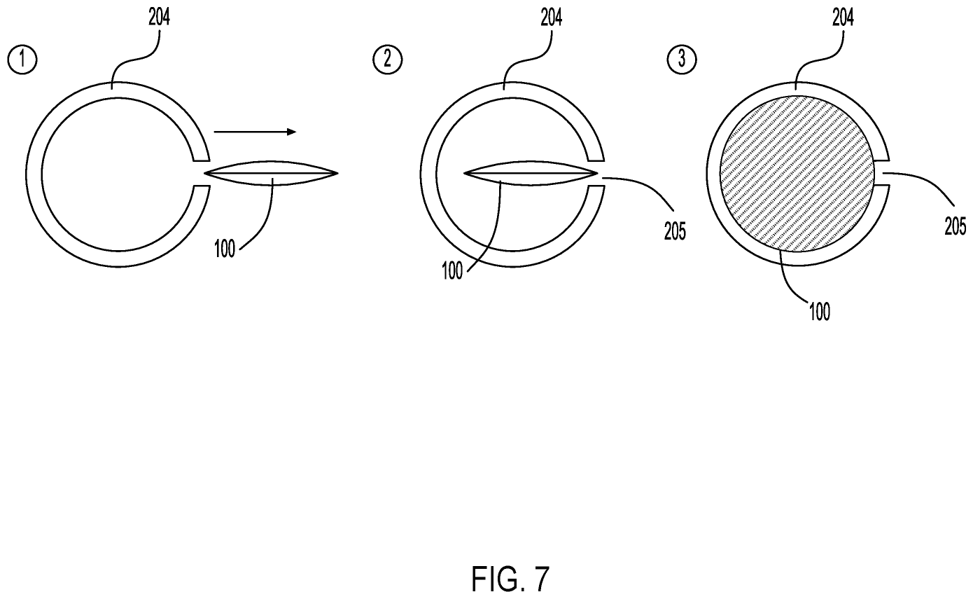

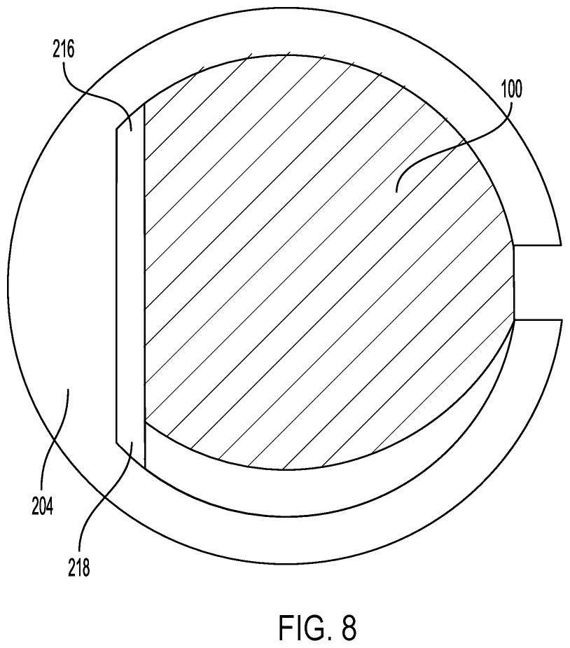

Some patients are tempted to remove their immobilization devices before their recommended treatment period. To mitigate this activity, an example embodiment proposes permanent attachments of the accessory to the lattice structure 100, all of which are applied to the lattice structure 100 before it is applied to the patient. As such, in one example, the coupling mechanism 204 is configured to be fixed to the lattice structure 100. In one such example, as shown in FIGS. 7-8, the coupling mechanism 204 comprises a rigid open ring structure with a fixed opening 205. The lattice structure 100 is configured to receive a liquid resin and a catalyst mixture that transforms into a solid such that the lattice structure hardens to thereby support the first joint of the body. The rigid open ring structure is configured to be positioned around the lattice structure 100 prior to the lattice structure 100 receiving the liquid resin and the catalyst mixture, as shown in the first two images of FIG. 7. A diameter of the lattice structure 100 is larger than the opening 205 of coupling mechanism 204 after receiving the liquid resin and the catalyst mixture such that the coupling mechanism 204 is permanently fixed to the lattice structure 100 once the liquid resin and the catalyst mixture transforms into a solid, as shown in the final image of FIG. 7.

Due to the order of operations, the attachments here do not need to be semi-rigid or flexible—rigid metal clasps can be pressed together around the edges of the parent device before patient application. The embodiments shown in FIGS. 7-8 describes the open-ring structure in embodiment, this embodiment specifically details that the inner diameter of the open-ring attachment is smaller than the outer diameter or radius of curvature of the parent device when in its fully rigid, applied state. As the parent device is applied, the material will expand around the attachment, locking it into place. As shown in FIG. 8, the coupling mechanism 204 may take advantage of a “D” shape 216 on the inside of the open-ring. This coupling type will prevent any rolling or sliding that may occur.

In a separate embodiment, the D edge may be coated in an additional material 218. This is to further prevent slipping of the ring that could cause it to slide up and down the edge of structure 100. This additional material will add friction between the smooth surface of the ring and the parent device. It could be a silicone, foam, or textured polymer coated deposited through dipping, spraying, or adhering.

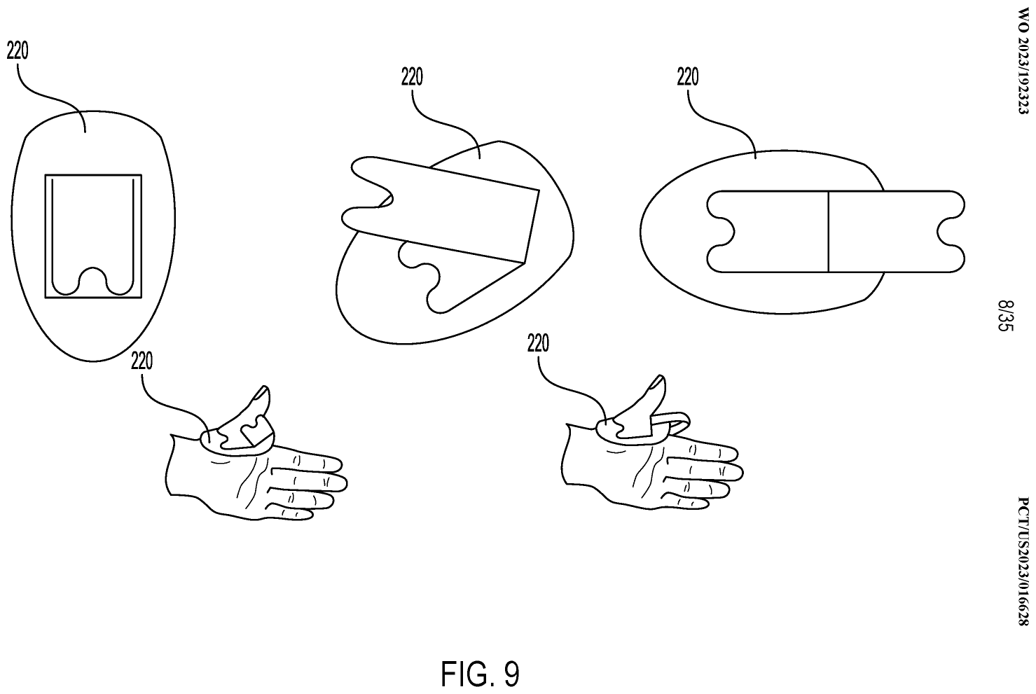

Any of these attachments could be used alone or in combination with each other for the proper immobilization of a secondary joint to create a modular immobilization device with a parent device with at least one accessory device. Additionally, any of these accessory devices can be manufactured with a padding layer to increase patient comfort. This can be applied in a pressure-sensitive adhesive, dip coating, or other method. For added comfort, a supplemental comfort lining as a prefabricated liner and cut to a shape for a body area or may be trimmed to accommodate various shapes of body areas may be used in conjunction with any of the aforementioned attachment mechanisms. Such a comfort liner 220 is illustrated in FIG. 9. This added comfort liner 220 is preferably applied to the patient before the lattice structure is applied to the patient.

In one example, the present disclosure provides a system including the lattice structure 100 and the device 200.

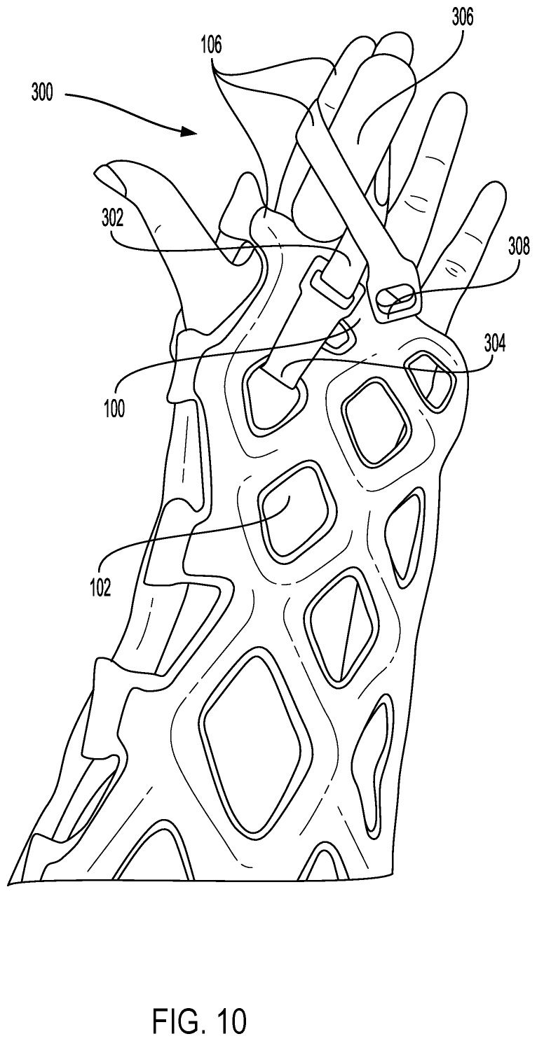

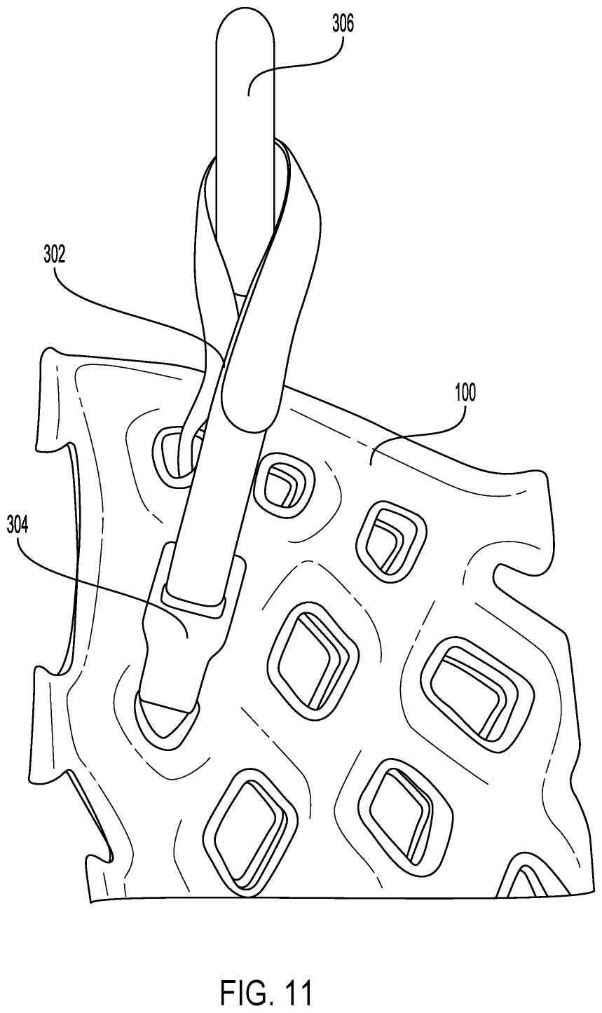

FIGS. 10-11 illustrate another device 300 configured to be coupled to a lattice structure 100. As described above, the lattice structure 100 is configured to support a first joint 104 of a body and the lattice structure 100 defines a plurality of apertures 102 configured to allow for a flow of air and water to the first joint 104. The device 300 includes a strap 302 having a first end and a second end opposite the first end. The device 300 also includes a coupling mechanism 304 coupled to the first end of the strap. The coupling mechanism 304 is configured to couple the device 300 to the lattice structure 100. The device 300 also includes an immobilization component 306 configured to be held in place by the strap 302. The immobilization component 306 is configured to support a second joint 106 of the body to thereby at least partially immobilize the second joint 106.

In one example, at least a portion of the strap 302 includes a hook and loop fastener. In one such example, the strap 302 is wrapped around the immobilization component 306 and then attached to itself to thereby secure the immobilization component 306 to the second joint 106. In one example, the immobilization component 306 is rigid.

In one example, the second end of the strap 302 includes a second coupling mechanism 308, and the second coupling mechanism 308 is configured to couple the second end of the strap 302 to the lattice structure 100. In one example, the coupling mechanism 304 comprises one of a t-anchor, an s-hook, or a c-hook.

The apertured lattice structure 100 lends itself to various strapping and hooks around its edges, as shown in FIGS. 10-11. In this embodiment, a rigid accessory for joint immobilization is secured to the lattice structure 100 through the strap 302. The length and shape of the rigid accessory will determine how many secondary joints will be immobilized. In one embodiment, the strap is secured around one edge of the parent device before the lattice structure 100 is applied to the patient. The strap is then wrapped around or through the accessory before attaching to itself or buckling or snapping closed, which can occur before or after the parent device is applied to the patient. In this embodiment, the shape of the accessory is a rectangle and long enough to secure three secondary joints in linear succession. This accessory can be a support or immobilizing device consisting of the same hardenable and rigid resinous material of the parent device. This is the embodiment for this accessory for immobilizing the thumb or digits in their position of function or with the digits fully flexed, where the strap 302 would be in tension.

In one example, the present disclosure provides a system including the lattice structure 100 and the device 300.

### B. Accessories

Secondary comorbidities are not uncommon with long-term immobilization. Specifically for the hand, muscle tone and overall dexterity can be lost during the weeks or months of immobilization. Proposed here are accessory attachments to improve a patient's ability to perform daily activities (such as grasping an eating utensil), continue productivity around the household (such as securely gripping a gardening shovel), or rehabilitate range of motion and strength using common physical and occupational therapeutic aides (such as squeezing a stress ball or pulling against resistance bands). Each accessory item is manufactured uniquely to loop, latch, or tie through a lattice of the rigid device, and wrap around the affected body area such that the accessory is securely fastened. The methods to secure the accessory aide around the affected body area to the rigid device may be fastened by but not limited to the following methods listed above (hook and loop fastener straps, t-anchors, elastic ties, c-hooks, s-hooks, etc.). The accessory aide latches are such that they are removable, adjustable, and replaceable. The accessories themselves include, but are not limited to, utensils, household tools, or rehabilitative equipment.

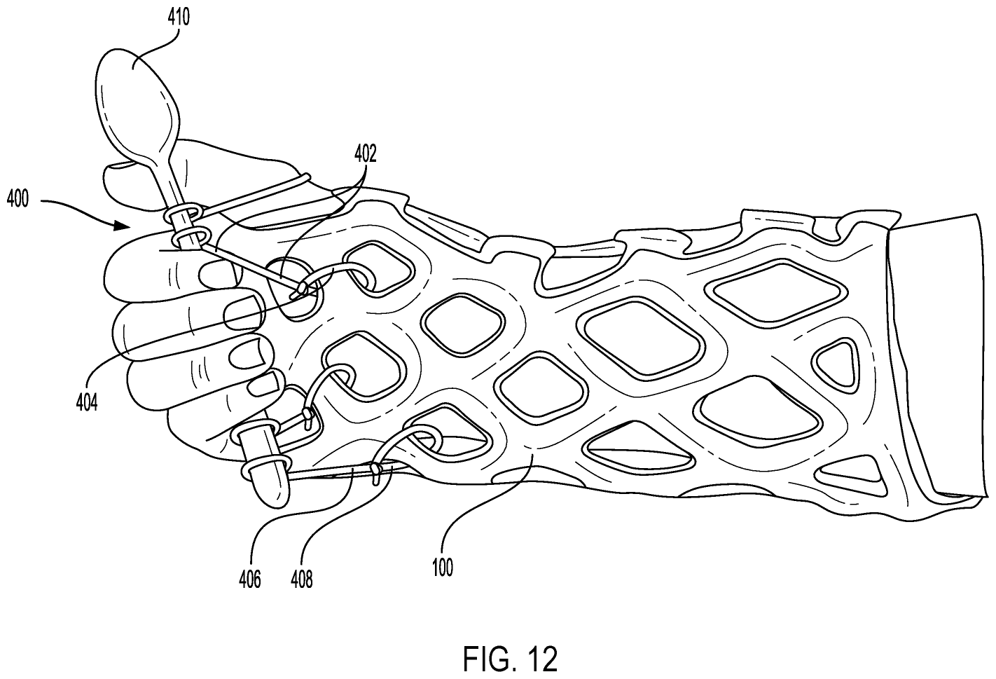

Accordingly, FIGS. 12-14 illustrate another device 400 configured to be coupled to a lattice structure 100. As described above, the lattice structure 100 is configured to support a first joint 104 of a body, and the lattice structure 100 defines a plurality of apertures 102 configured to allow for a flow of air and water to the first joint 104. The device 400 includes a first elastic cable 402 having a first end and a second end opposite the first end. The device 400 also includes a first coupling mechanism 404 coupled to the first end of the first elastic cable 402. The first coupling mechanism 404 is configured to couple the first elastic cable 402 to a first aperture of the plurality of apertures 102 of the lattice structure 100. The device 400 also includes a second elastic cable 406 having a first end and a second end opposite the first end. The device 400 also includes a second coupling mechanism 408 coupled to the first end of the second elastic cable 406. The second coupling mechanism 408 is configured to couple the second elastic cable 406 to a second aperture of the plurality of apertures 102 of the lattice structure 100. The second end of the first elastic cable 402 and the second end of the second elastic cable 406 are configured to hold a utensil 410. In one example, the first coupling mechanism 404 and the second coupling mechanism 408 comprise one of a t-anchor, an s-hook, or a c-hook.

In one example, the device 400 further includes a third coupling mechanism positioned at the second end of the first elastic cable 402 and a fourth coupling mechanism positioned at the second end of the second elastic cable 406, where the third coupling mechanism and the fourth coupling mechanism are configured to hold the utensil 410.

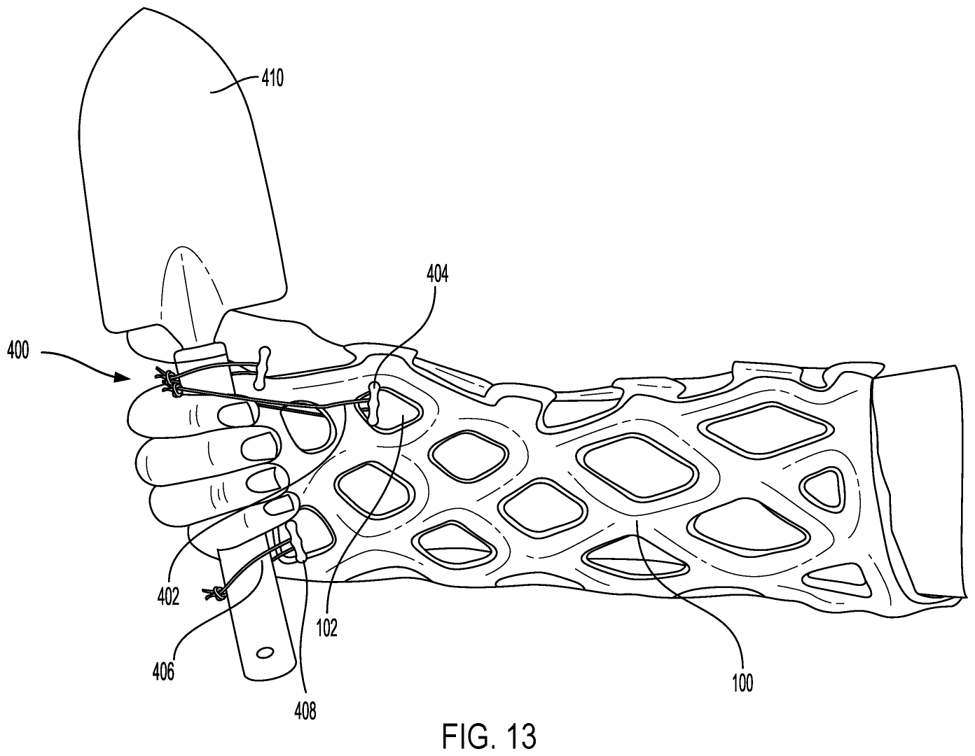

The embodiment shown in FIG. 12 describes a looping mechanism to hold a utensil such as a spoon. Elastic loops are secured to the lattice structure 100 and around the utensil 410. These straps with loops on both ends are in constant tension to ensure the utensil is close to the wearer's body. In one embodiment, FIG. 13 shows a shovel tool secured to the parent device through two t-anchors on an elastic loop. This attachment mechanism has one attachment point of the parent device and one to itself, allowing for the tool to be dropped from the wearer's grip without falling. FIG. 14 is an example of hook and loop strapping to secure a compression orb to the parent device by wrapping around the orb accessory and then around the lattice structure 100 without being secured to any edges. This embodiment is specifically preferred for rehabilitative accessories intended to increase dexterity, where edge attachments may be more difficult to apply.

In one example, the present disclosure provides a system including the lattice structure 100 and the device 500.

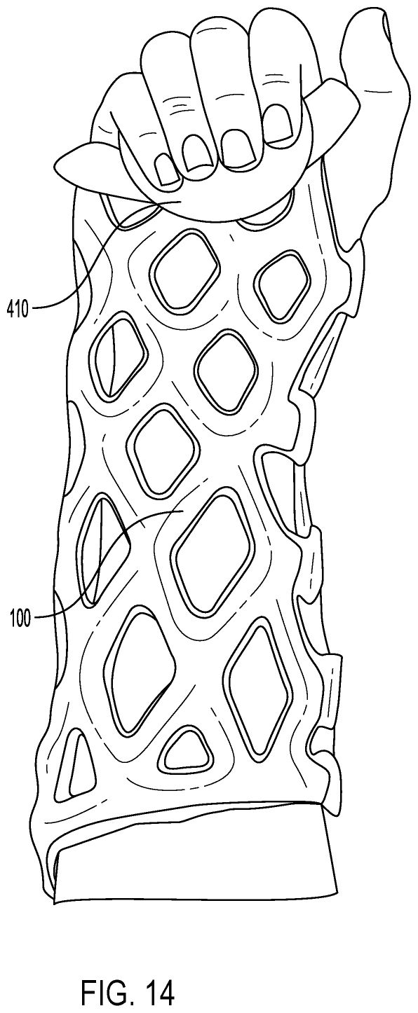

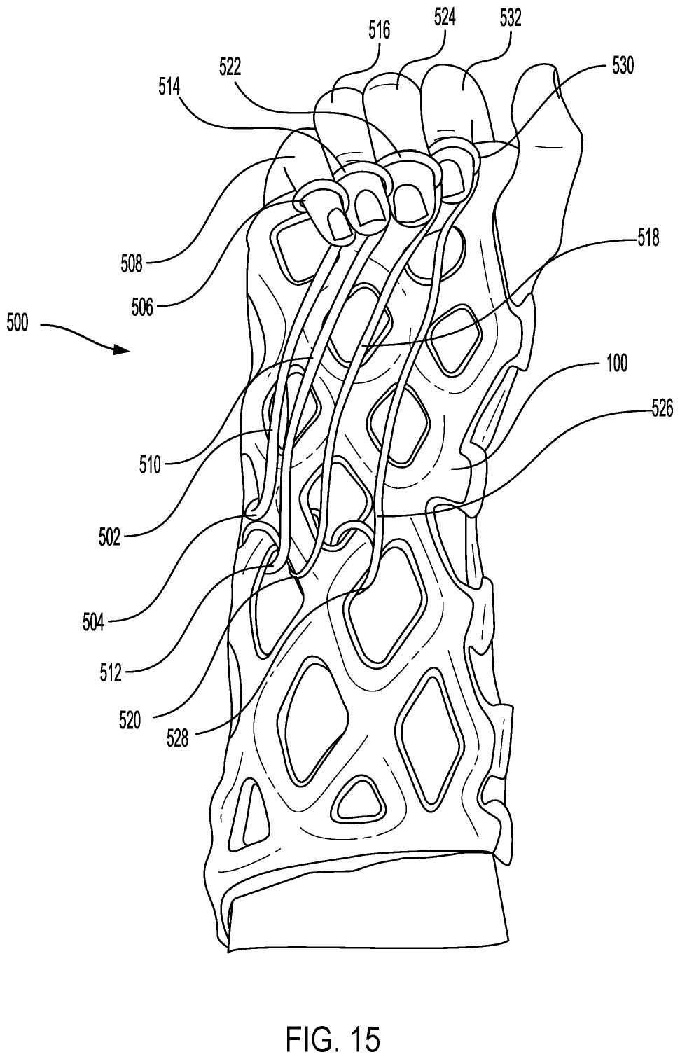

FIG. 15 illustrates another device 500 configured to be coupled to a lattice structure 100. As described above, the lattice structure 100 is configured to support a first joint 104 of a body, and the lattice structure 100 defines a plurality of apertures 102 configured to allow for a flow of air and water to the first joint 104. The device 500 includes a first elastic cable 502 having a first end and a second end opposite the first end. The device 500 also includes a first coupling mechanism 504 positioned at the first end of the first elastic cable 502. The first coupling mechanism 504 is configured to couple the first elastic cable 502 through an aperture of the plurality of apertures 102 of the lattice structure 100. The device 500 also includes a second coupling mechanism 506 positioned at the second end of the first elastic cable 502. The second coupling mechanism 506 is configured to couple the first elastic cable 502 to a first digit 508 of the body. The device 500 also includes a second elastic cable 510 having a first end and a second end opposite the first end. The device 500 also includes a third coupling mechanism 512 positioned at the first end of the second elastic cable 510. The third coupling mechanism is 512 configured to couple the second elastic cable 510 to an aperture of the plurality of apertures 102 of the lattice structure 100. The device 500 also includes a fourth coupling mechanism 514 positioned at the second end of the second elastic cable 510. The fourth coupling mechanism 514 is configured to couple the second elastic cable 510 to a second digit 516 of the body. The device 500 also includes a third elastic cable 518 having a first end and a second end opposite the first end. The device 500 also includes a fifth coupling mechanism 520 positioned at the first end of the third elastic cable 518. The fifth coupling mechanism 520 is configured to couple the third elastic cable 518 to an aperture of the plurality of apertures 102 of the lattice structure 100. The device 500 also includes a sixth coupling mechanism 522 positioned at the second end of the third elastic cable 518. The sixth coupling mechanism 522 is configured to couple the third elastic cable 518 to a third digit 524 of the body. The device 500 also includes a fourth elastic cable 526 having a first end and a second end opposite the first end. The device 500 also includes a seventh coupling mechanism 528 positioned at the first end of the fourth elastic cable 526. The seventh coupling mechanism 528 is configured to couple the fourth elastic cable 526 to an aperture of the plurality of apertures 102 of the lattice structure 100. The device 500 also includes an eighth coupling mechanism 530 positioned at the second end of the fourth elastic cable 26. The eighth coupling mechanism 530 is configured to couple the fourth elastic cable 526 to a fourth digit 532 of the body.

The device 500 described above is an accessory for increasing muscle tone and maintaining tendon flexibility of the digits during wrist immobilization. In one embodiment, the attachment system includes c-hooks which can be easily applied over the edge of the lattice structure 100. Elastic loops as the accessory dangle from this attachment, and the digits can pull the loops into various degrees of tension. Further, these modular accessories can be interchanged for loops with differing elastic properties to increase or decrease the difficulty of pulling the loop into tension.

In one example, the present disclosure provides a system including the lattice structure 100 and the device 500.

FIG. 16 illustrates another device 600 configured to be coupled to a lattice structure 100. As described above, the lattice structure 100 is configured to support a first joint 104 of a body, and the lattice structure 100 defines a plurality of apertures 102 configured to allow for a flow of air and water to the first joint 104. The device 600 includes a through-hole 602 positioned at the first end of the device 600. As shown in FIG. 16, the through-hole 602 is configured to receive at least a portion of a thumb of the body when in use. In another example, the device 600 does not include a through-hole and instead the thumb rests within a notch 608 of the device 600 when the device 600 is in use. The device 600 further includes a utensil 604 positioned at a second end of the device 600 opposite the first end. In one example, the utensil 604 positioned at the second end of the device 600 comprises one of a spoon, a fork, a knife, or any other utensil for non-eating purposes. The device 600 also includes an attachment mechanism 606 configured to be removably positioned at least partially within an aperture of the plurality of apertures 102 of the lattice structure 100.

In one example, the attachment mechanism comprises a notch 608 and a protrusion 610 configured to contact an edge of the aperture 102 to thereby prevent slipping or turning of the device when in use. Made of a semi-flexible, rigid material, the notch 608 and the protrusion 610 are used to pair with the predetermined shape and structure of the aperture 102 in the lattice structure 100. The notch 608 and the protrusion 610 in the device 600 provide a single form-factor in which the accessory is the attachment mechanism. When the device 600 is gently pressed against at least one edge or intersection of the lattice structure 100, the sides of the protrusion 610 contract and then expand to its original dimensions, holding at least one edge of the lattice structure 100 or at least one intersection or any combination of edge and intersection in compression, as shown in FIG. 16. In addition, the protrusion 610 positioned within the aperture 102 of the lattice structure 100 will prevent slipping or turning. This design prevents dropping of the device 600. Unlike previous descriptions of utensil attachments, this embodiment eliminates the need for the wearer to grip the device at all.

In one example, the present disclosure provides a system including the lattice structure 100 and the device 600.

### C. Multi-Modular Systems

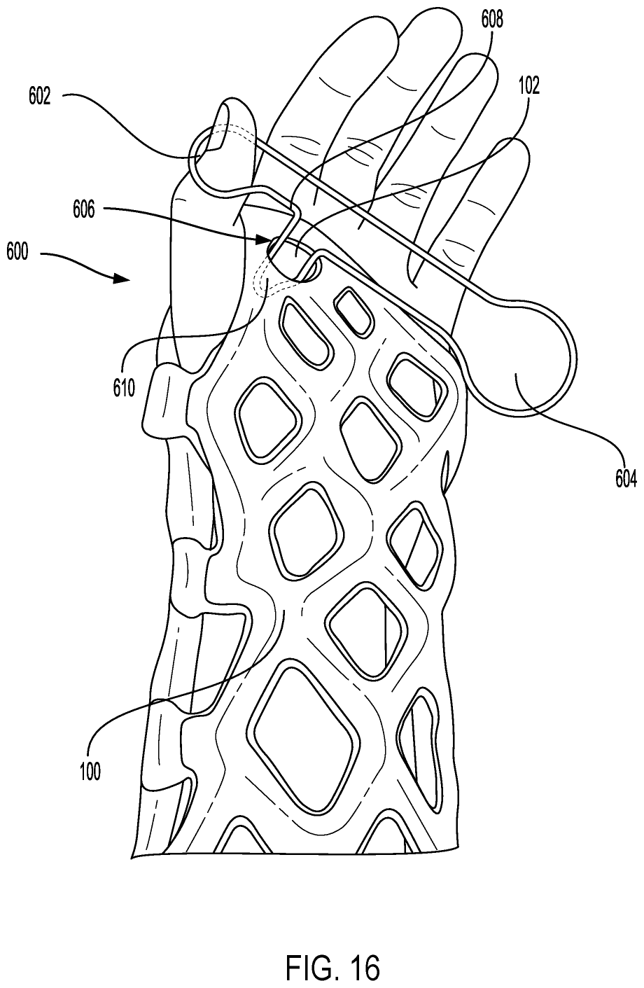

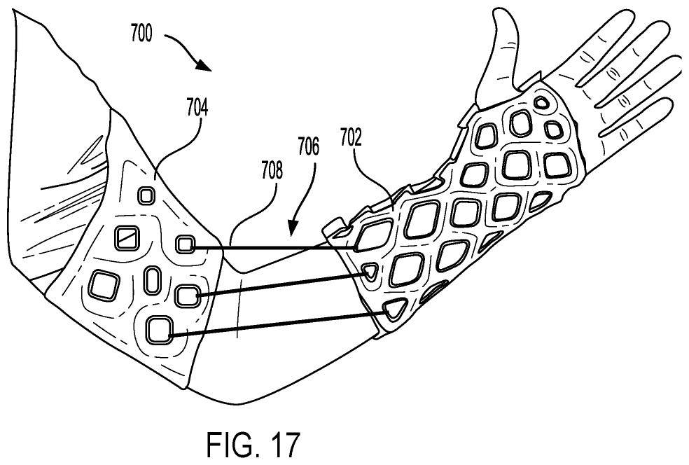

FIGS. 17-31 illustrate a variety of multi-modular assembly systems that allow for the immobilization of more than one joint or affected area, with a mechanism that connects the modules or components. FIGS. 17-31 further illustrate methods of securing multiple sections of a single component to create a cylindrical sleeve.

Accordingly, in one example the present disclosure provides a system 700 including a first lattice structure 702 configured to support a first area of a body. The first lattice structure 702 defines a first plurality of apertures configured to allow for a flow of air and water to the first area of the body. The system 700 further includes a second lattice structure 704 configured to support a second area of the body. The second lattice structure 704 defines a second plurality of apertures configured to allow for a flow of air and water to the second area of the body. The system 700 further includes a coupling mechanism 706 configured to couple the first lattice structure 702 to the second lattice structure 704.

In one example, as shown in FIG. 17, the coupling mechanism comprises a plurality of struts 708 each having a first end and a second end opposite the first end. The first end of each of the plurality of struts 708 are coupled to the first plurality of apertures of the first lattice structure 702, and the second end of each of the plurality of struts are coupled to the second plurality of apertures of the second lattice structure 704.

In this embodiment, the connection between first lattice structure 702 and the second lattice structure 704 which are not in contact with each other, is achieved through the use of a rigid strut that is secured as a loop or hook over or under the edges or intersections of the modules. One or more attachments can be used for a single system to secure the struts and ensure adequate immobilization of the joint.

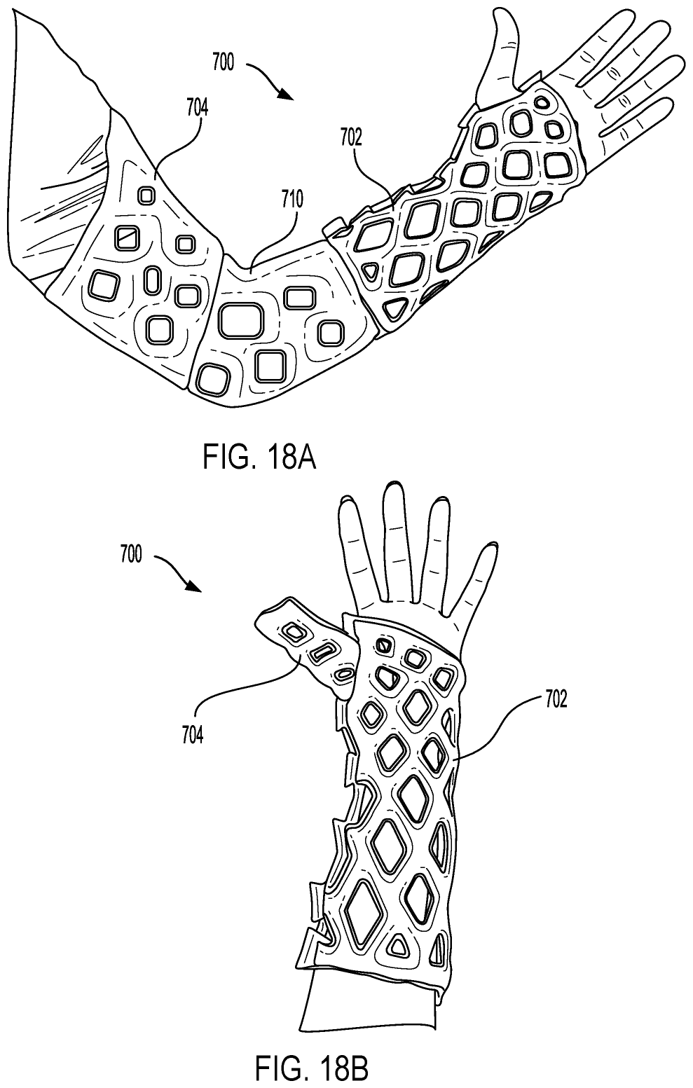

In one example, as shown in FIG. 18A, the system 700 further includes a third lattice structure 710 configured to support a third area of the body. The third lattice structure 710 defines a third plurality of apertures configured to allow for a flow of air and water to the third area of the body. In such an example, the system 700 may further include a second coupling mechanism configured to couple the second lattice structure 704 to the third lattice structure 710. FIG. 18A illustrates the first lattice structure 702 connected to second lattice structure 704 with the third lattice structure 710 providing contact surfaces to establish a complete connection between components.

In another example, as shown in FIG. 18B, the system 700 and the coupling mechanism 706 providing the coupling between the first lattice structure 702 and the second lattice structure 704 is a compression or press fit. As shown in FIG. 18B, in one example a portion of the second lattice structure 704 is wedged between a portion of the first lattice structure 702 and the skin of the patient. The compressive force between the first lattice structure 702 and the skin of the patient holds the second lattice structure 702 in place. In one example, the second lattice structure 704 is initially positioned on the desired joint, and then the a liquid resin and a catalyst mixture are injected into a lumen of the first lattice structure 702 to thereby harden and apply the compressive force to hold the second lattice structure 704 in place.

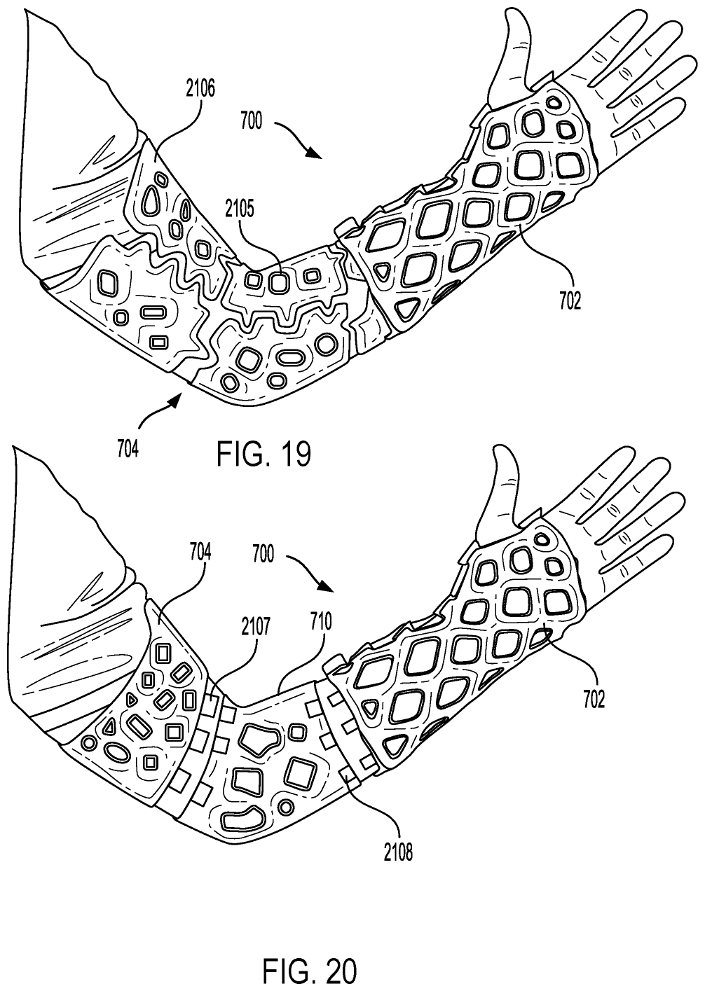

The example shown in FIG. 19 illustrates the second lattice structure 704 comprises multiple male rigid modules 2106 and female rigid modules 2105 creating a locking mechanism at the points of contact by fitting them together in place. Such a structure may also be used to fit the second lattice structure 704 to the first lattice structure 702. The components are connected together by using the rigid outer edges of the components as a locus of pressure points that hold the modules together.

The example shown in FIG. 20 illustrates the first lattice structure 702 and the second lattice structure 704 including hollow male protrusions 2107 snapped together through hollow female protrusions 2108 on the joining third lattice structure 710. The snapping mechanism creates a flowing system across multiple modules. In this embodiment, the components consist of a series of male and female sections along the outer edges. The female components and male components may consist of protrusions or depressions. The components are then press-fitted together by using the rigid edges and the male and female sections as pressure points that hold the modules together. In one example, each of the hollow male protrusions 2107 and/or each of the hollow female protrusions 2108 include a locking mechanism to prevent flow through those components. In one example, the locking mechanism comprises a check valve that only allows for flow between the hollow male protrusions 2107 and the hollow female protrusions 2108 when they are connected to one another. In another example, the locking mechanism comprises a ball valve. In another example, the locking mechanism comprises a butterfly valve. Other locking mechanisms are possible as well.

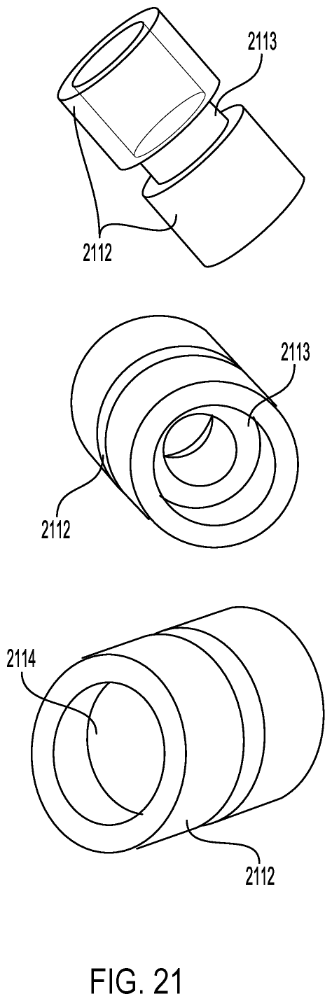

FIG. 21 illustrates a first view showing male protrusions 2112 and a hollow female protrusion 2113 to establish a multi-modular connection which allows flow of liquid between components, a second view of male protrusions 2112 and a hollow female protrusion 2113 to establish a multi-modular connection which allows flow of liquid between components, and a third view showing male protrusions 2112 and a solid female protrusion 2114 to establish a multi-modular connection that prevents flow of liquid between components. In one example, the connection between two or more components or modules, which are in contact with each other, is achieved using a male and female snapping or press-fitting mechanism. The female components consist of protrusions with a larger cross section, the male components also consist of protrusions with a smaller cross section. In another example, the protrusions on both the male and female components are hollow and they provide a passage for flow of liquid between components allowing multiple modules to combine into a single form factor. In another example, the protrusions on the male components are hollow while the protrusions on the female component are solid, preventing flow of liquid between components. The resulting system behaves as a rigid structure while being filled with liquids separately.

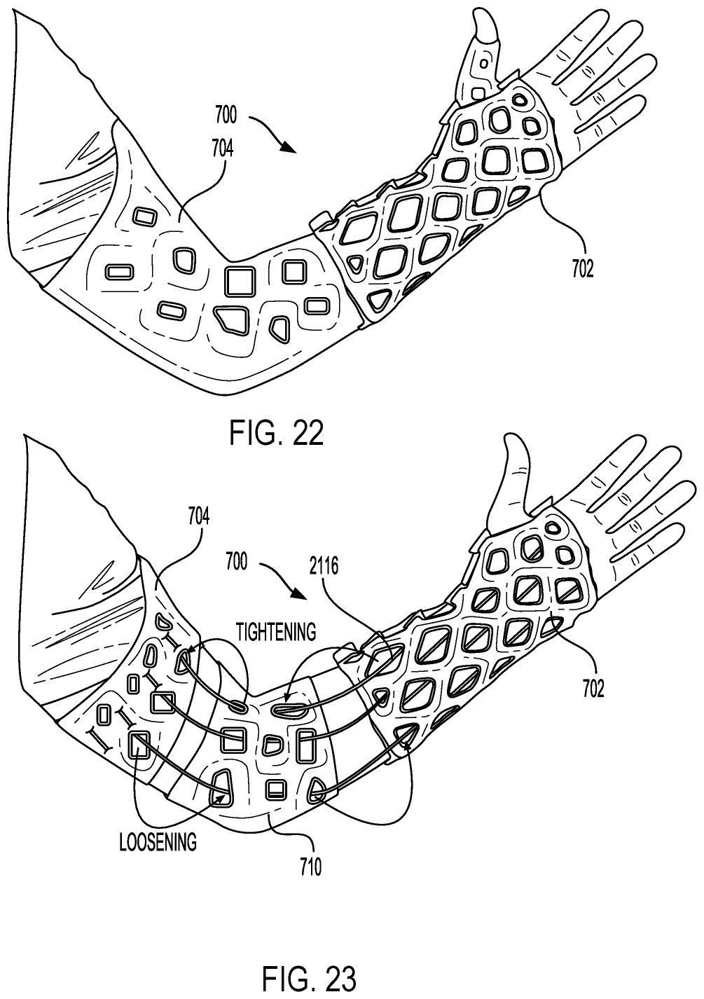

FIG. 22 illustrates a single form factor sleeve for the second lattice structure 704 that creates a rigid structure that provides immobilization across multiple joints. In this example, the immobilization of two or more joints is achieved by using a single component that acts as a singular parent device across multiple joints. FIG. 23 illustrates the first lattice structure 702 connected to the second lattice structure 704 with a central third lattice structure 710, all suspended on a flexible string 2116, which are tightened or loosened to bring the components together, allowing immobilization and support at the joint. In this embodiment, the connection between two or more components is achieved by connecting the components through semi-rigid strings on which the components are suspended. The suspended components are pulled into position and are tightened by using an external tool. In another example, the suspended components are pulled into position and are tightened manually.

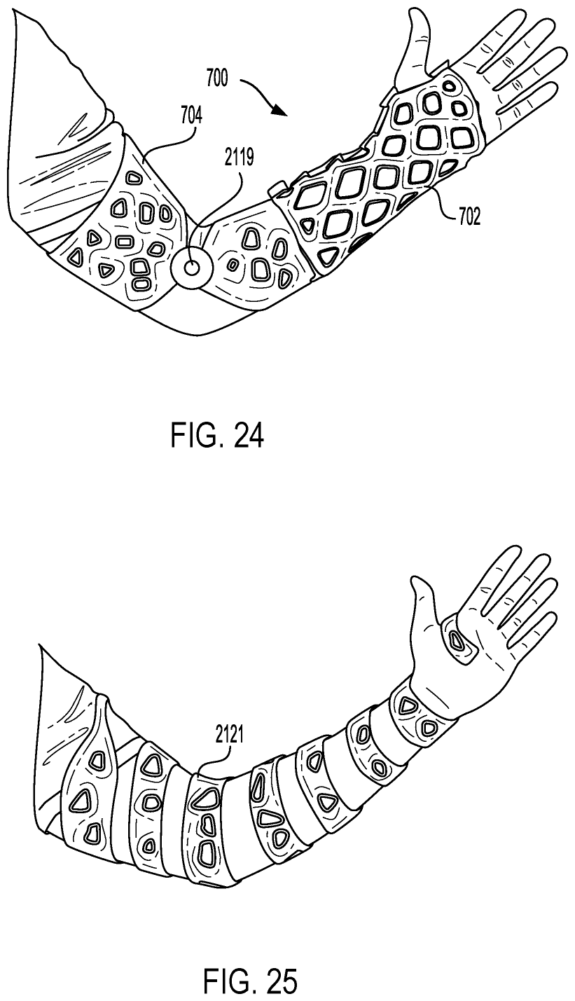

FIG. 24 illustrates the first lattice structure 702 connected to the second lattice structure 704 through a circular central knob or dial 2119, which brings the components together under tension when turned clockwise and releases tension when turned counterclockwise.

FIG. 25 illustrates a 3-layer sleeve 2121 wrapped on the arm across multiple joints, which when rigid allows for immobilization of joints. In this embodiment, there is a singular component consisting of a sleeve with multiple layers, rolled on a cylindrical base. The component is applied by unrolling and wrapping it around the affected area before being filled with liquid. The filled sleeve provides orthopedic support upon becoming rigid.

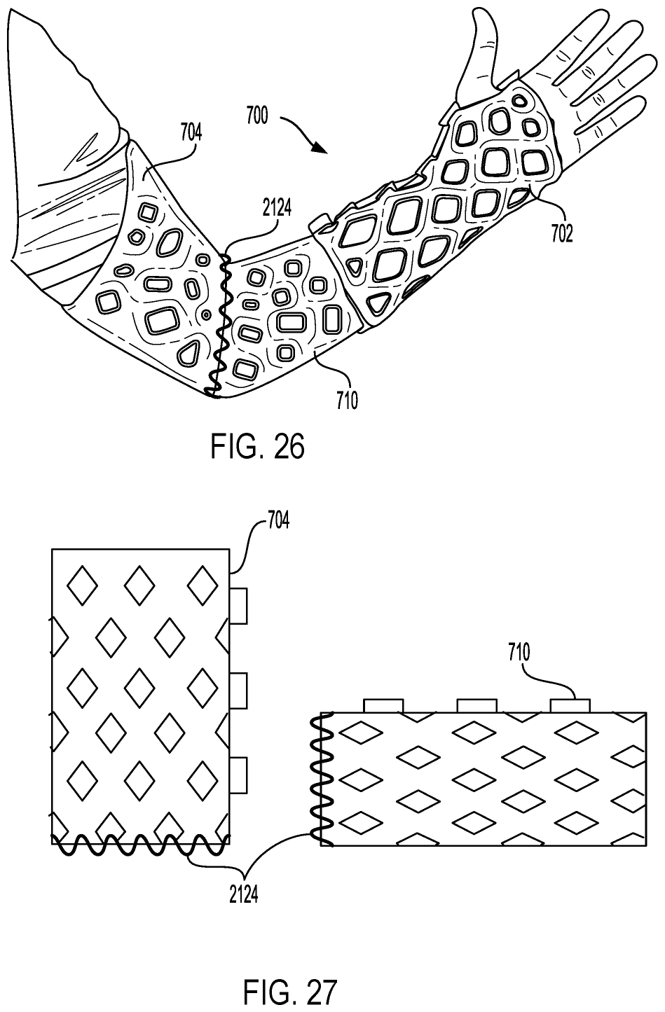

FIG. 26 illustrates the second lattice structure 704 connected to the third lattice structure 710 through the use of a pairing mechanism 2124 between the edges in contact. In one example, the pairing mechanism 2124 may comprise a zipper. In another example, the pairing mechanism 2124 may comprise a hook and loop fastener. FIG. 27 illustrates a pairing mechanism 2124 attached to the lateral edges of the second lattice structure 704 and the third lattice structure 710. In this embodiment, the connection between two or more components is achieved through the use of a pairing mechanism attached to the edges of the components. A central pairing mechanism is used to pull the components together creating a rigid structure at the joint.

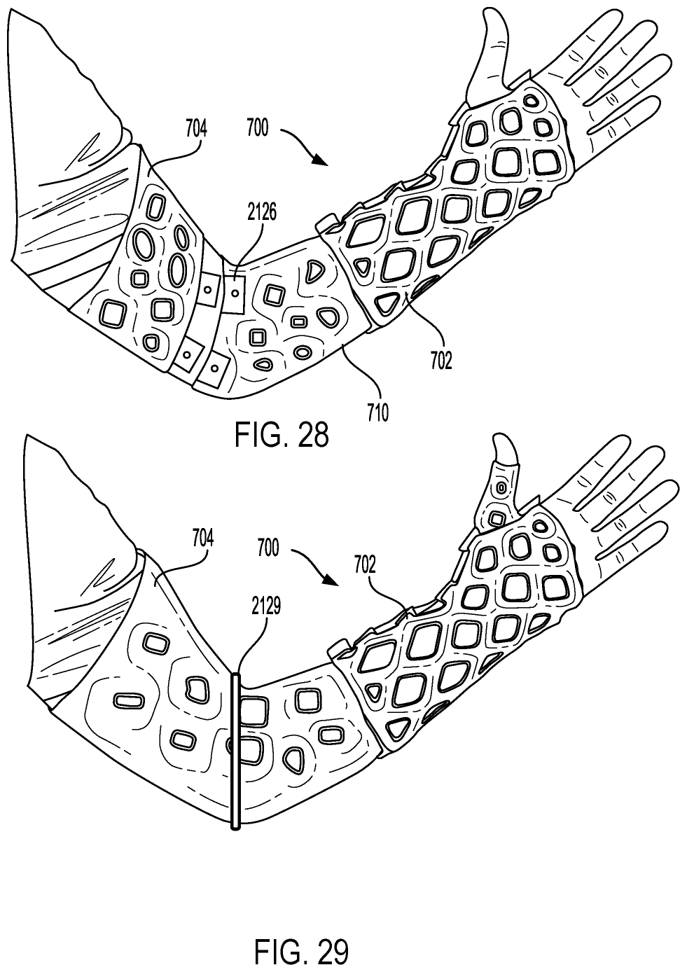

FIG. 28 illustrates an adhesive section 2126 placed on a male protrusion on the second lattice structure 704 and a female protrusion on the third lattice structure 710. The adhesive usually used is liquid and hardens upon curing creating a strong bond that results in stable connection between the components.

FIG. 29 illustrates with a single form factor as the second lattice structure 704 with a clip 2129 placed at the section covering the joint before liquid being filled. Upon hardening of the liquid, the clip is removed creating a bendable flexible zone at the joint. In this embodiment, the immobilization of two or more joints is achieved by using a single component that acts as a singular parent device across multiple joints and the immobilization around a joint is achieved by the use of restraining clips creating regions which are flexible in a structure which is rigid, allowing for setting in zones where bends are present.

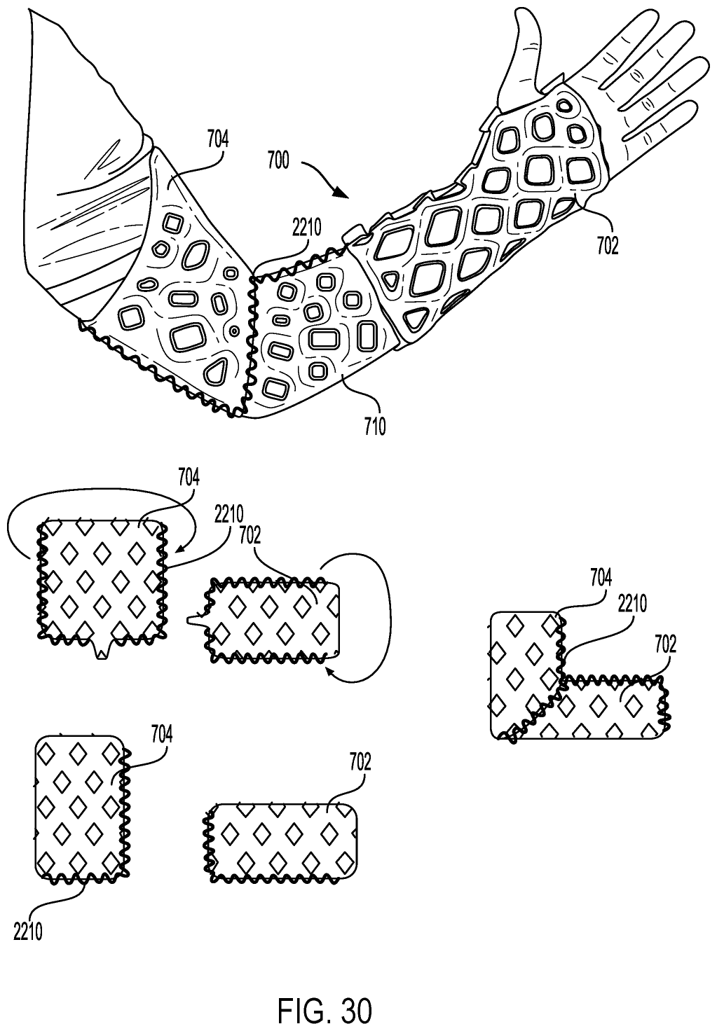

FIG. 30 illustrates the second lattice structure 704 and the third lattice structure 710 sealed together through the use of a pairing mechanism 2210 across the lateral and axial edges of the components, which connects the components and also allows the cylindrical sealing of a flexible sleeve prior to being filled with liquid. As discussed above, in one example, the pairing mechanism 2210 may comprise a zipper. In another example, the pairing mechanism 2210 may comprise a hook and loop fastener. Upon hardening, the pairing mechanism 2210 makes the product a removable device.

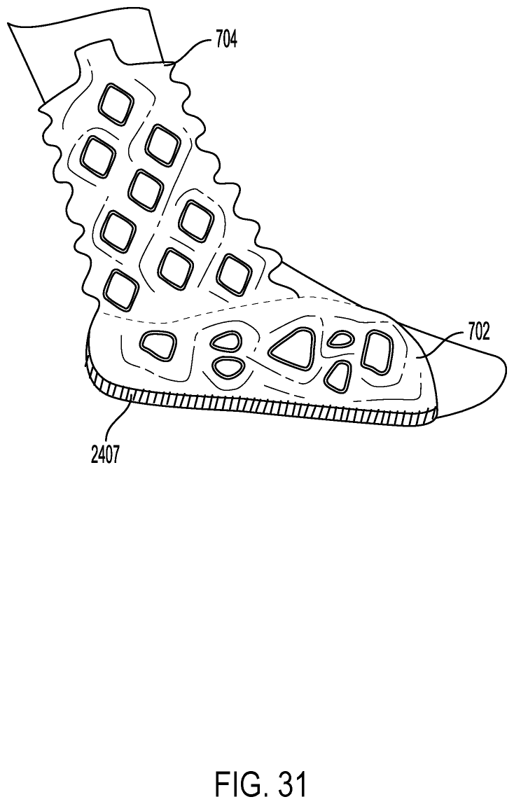

FIG. 31 illustrates a weight bearing semi-rigid base 2407 attached to the first lattice structure 702, which is attached to the second lattice structure 704 through stitches, snaps or welds. In one example, an anterior section of the semi-rigid base 2407 comprises a foam material to provide therapeutic relief. In this embodiment, a two-component system consists of a load bearing component (first lattice structure 702) on which the weight of the system rests. The other component which is not load bearing (second lattice structure 704) is attached to the load bearing component by snapping the components together, or through the use of stitches or welds as non-limiting examples.

### D. Coupling Systems

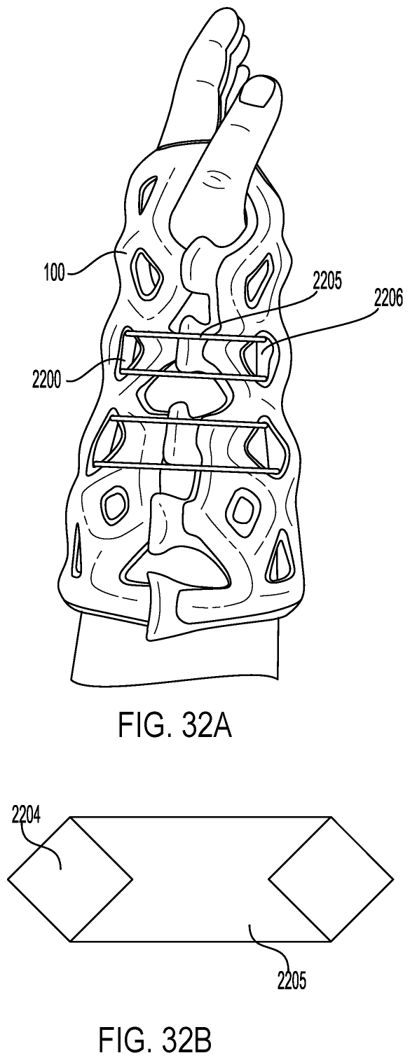

With reference to FIGS. 32A-36, the present disclosure provides a variety of methods and systems for securing the lattice structure 100 during application to a body area.

In one example, the present disclosure provides a method of forming a support for application to a body area. The method may include (i) positioning a lattice structure around the body area, (ii) coupling a first portion of the lattice structure to a second portion of the lattice structure via a coupling mechanism, and (iii) injecting a liquid resin and a catalyst mixture into a lumen of the lattice structure, wherein the liquid resin and the catalyst mixture transforms into a solid in the lumen of the lattice structure such that the lattice structure hardens into the support for application to the body area.

In one example, as shown in FIG. 32A, the coupling mechanism comprises a locking strap 2205 having a first protrusion configured to be positioned within a first aperture 2200 of the first portion of the lattice structure 100 and a second protrusion configured to be positioned within a second aperture 2206 of the second portion of the lattice structure. As such, with the locking strap 2205 connects the left and right sections of the sleeves, and the protrusions of the locking strap 2205 fit and snap into the space on either side of the sleeve. In this embodiment, the component is secured as a cylindrical sleeve through the use of a locking strap made of a flexible material. The strap consists of protrusions that resemble the shape of the respective space or lattice on the right and left of the component. The protrusions are snapped and fit to secure the component. As shown in FIG. 32B, a locking strap 2205 connects the left and right sections of the sleeves, with the protrusions 2204 snapped into the apertures on the sleeve. In this embodiment, the component is secured as a cylindrical sleeve through the use of a locking strap made of a flexible material. The strap includes a first end and a second end opposite the first end. The first end of the strap includes a first protrusion that is complementary the shape of the respective space or lattice on the left side of the lattice structure 100, and the second end of the strap includes a second protrusion that is complementary the shape of the respective space or lattice on the right side of the lattice structure 100. The protrusions are snapped and fit to secure the component.

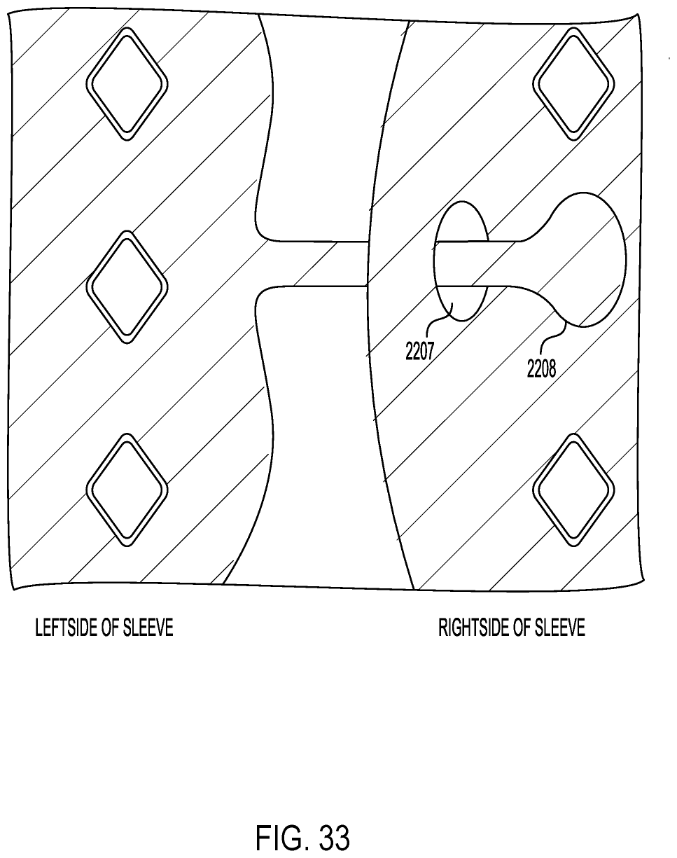

In another example, as shown in FIG. 33, the second portion of the lattice structure includes a through-hole 2207, and the first portion of the lattice structure includes an extension 2208 configured to be positioned through the through-hole, wherein a diameter of the extension is greater than a diameter of the through-hole after the liquid resin and the catalyst mixture transforms into a solid. In this embodiment, the component consists of extensions on the left which are part of the sleeve, and negative spaces on the right. The extensions are fitted into the negative spaces to be filled with liquid. Upon becoming rigid, the resin filled inside the extension secures the component in position.

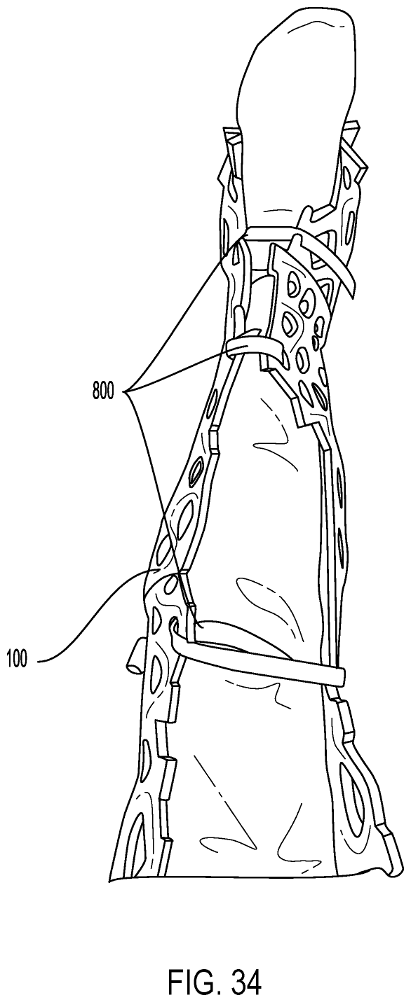

In another example, as shown in FIG. 34, the coupling mechanism comprises a plurality of hook and fastener straps 800. In such an example, the lattice structure 100 can be used to stabilize more than one joint by holding at least two joints immobile in at least two degrees of freedom but not circumferentially restricting and then secured using strapping mechanisms as described above. The strapping mechanism would not secure an accessory to the lattice structure 100, but would rather hold the parent device in position through compression. In one example, the straps 800 are secured to the lattice structure 100 via the apertures in the lattice structure. In an alternate embodiment, the straps 800 are attached to the lattice structure 100 via a section of excess material on the seams of the lattice structure 100. In an alternative embodiment, the straps 800 are secured to themselves and held in place by compression.

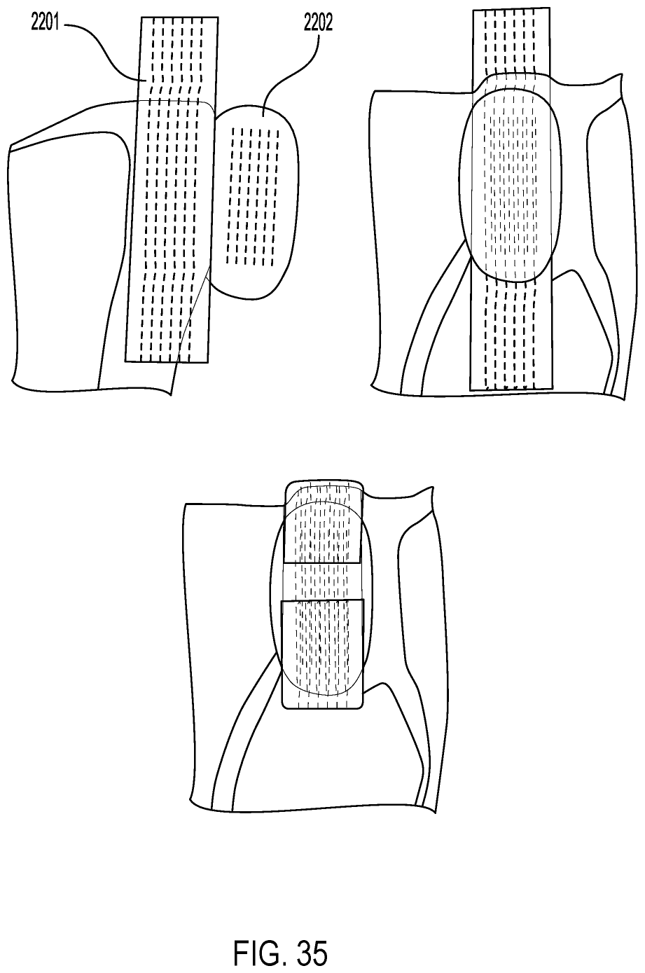

FIG. 35 illustrates another mechanism for securing the first portion of the lattice structure 100 to the second portion of the lattice structure 100. As shown in FIG. 35, the left tab containing an extended strip containing grooves 2201 and the right tab containing a short strip containing protrusions 2202, which are folded and sealed together by mating the grooves. The extended section is then folded on top of the short strip to create an axial lock. The upper left figure of FIG. 35 illustrates the left tab containing an extended strip containing grooves 2201 and the right tab containing a short strip containing protrusions 2202. The upper right figure of FIG. 35 illustrates the left and right tabs mated together through the grooves. The lower figure of FIG. 35 illustrates the left and right tabs mated together and sealed with the extended strip to create an axial lock. In this embodiment, the tabs present on the components are attached with a strip containing grooves, made of a semi-rigid material. The outside of the tabs on the left consist of an extended attachment. Both the inside and outside of the tabs on the right consist of an attachment which does not extend beyond the length of the tabs. The attachment on the inside of the tab on the right is snapped onto the extended attachment on the outside of the tab on the left. The extensions of the attachment on the outside of the tab on the left are then folded over and snapped onto the attachment on the outside of the tab on the right. In another example, the grooves 2201 and the protrusions 2202 comprise a zig-zag pattern to prevent longitudinal slipping between the components. In another example, the grooves 2201 and the protrusions 2202 comprise a circular shape to prevent longitudinal slipping between the components. Other arrangements are possible as well.

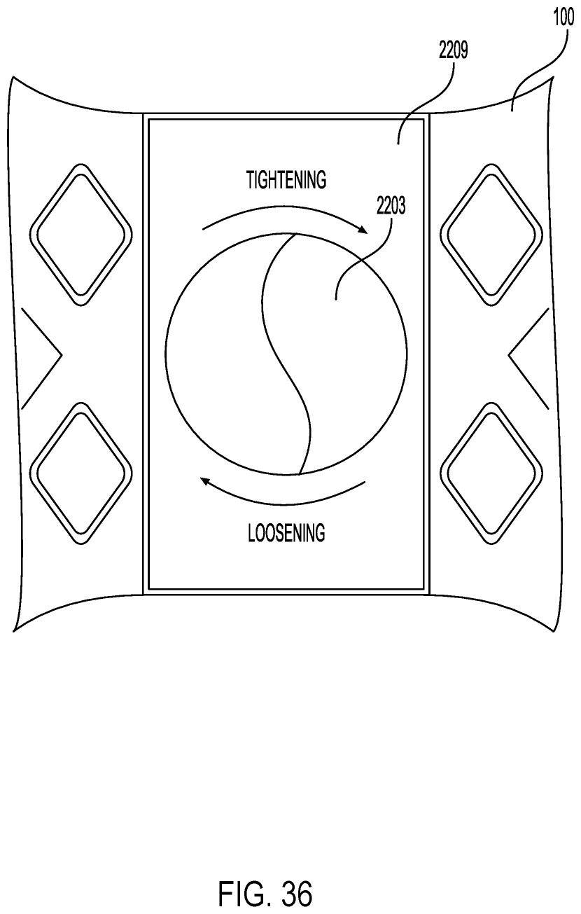

FIG. 36 illustrates a dial 2203 welded onto a tab 2209 created from two tabs (one from each side of the lattice structure 100) welded over each other. In such an example, the lattice structure 100 can be tightened by turning the dial 2203 clockwise bringing the edges of the lattice structure 100 closer, and the lattice structure 100 be loosened by turning the dial 2203 counterclockwise. In one embodiment, the tabs present on the opposing portions of the sleeve that make up the lattice structure 100 are sealed together with the central circular dial 2203. In another example, the dial 2203 is positioned on one of the tabs prior to welding the two tabs together.

### E. Flexible Zone of Lattice Structure

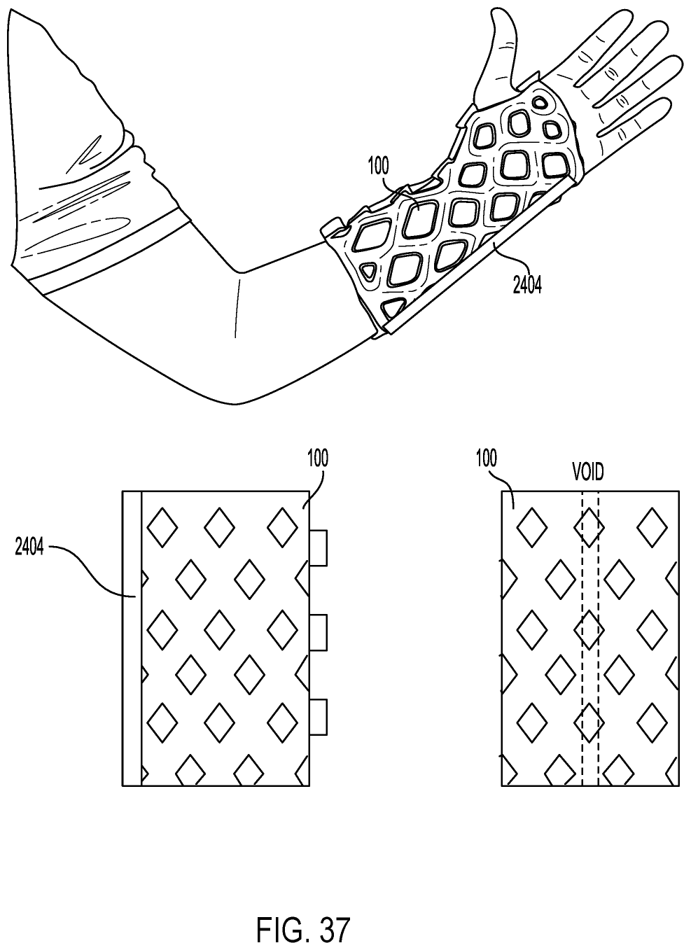

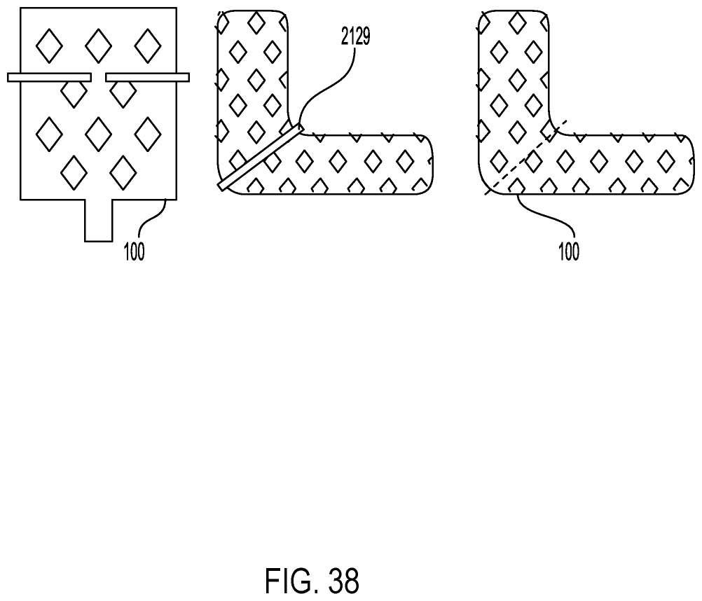

With reference to FIGS. 37-38, the present disclosure provides a variety of methods and systems for providing a flexible zone to the lattice structure 100 during application to a body area.

The present disclosure further provides another method of forming a support for application to a body area. The method comprises (i) positioning a lattice structure around the body area, (ii) coupling a restraining clip to a portion of the lattice structure, and (iii) injecting a liquid resin and a catalyst mixture into a lumen of the lattice structure, wherein the liquid resin and the catalyst mixture transforms into a solid in the lumen of the lattice structure such that the lattice structure hardens into the support for application to the body area, and wherein the restraining clip prevents the liquid resin and the catalyst mixture from entering a portion of the lumen thereby creating a void in the lumen. In one example, the void in the lumen creates a flexible section of the lattice structure.

FIG. 37 illustrates a lattice structure 100 containing a restraining clip 2404 which is removed upon hardening of the liquid, creating a flexible section at the void, allowing for easy removal and reuse of the components when required. In this embodiment, a restraining clip is placed on the cross section of the sleeve to create a region of the unfilled component. Upon filling and removal of the clip, the void region can be used to allow for the temporary removal of the product from the affected area and replacement it if needed. FIG. 38 illustrates a single form factor as a lattice structure 100 with a clip 2129 placed at the section covering the joint before liquid being filled. Upon hardening of the liquid, the clip is removed creating a bendable flexible zone at the joint.

### F. Injection System

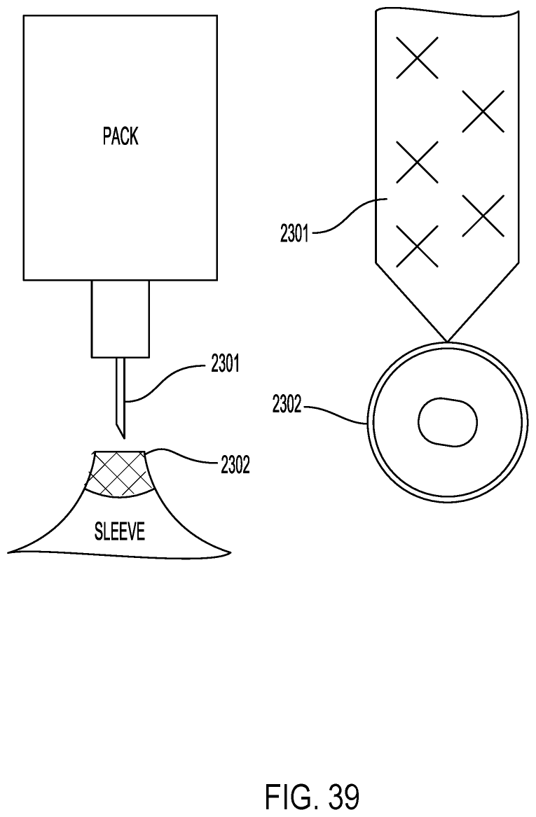

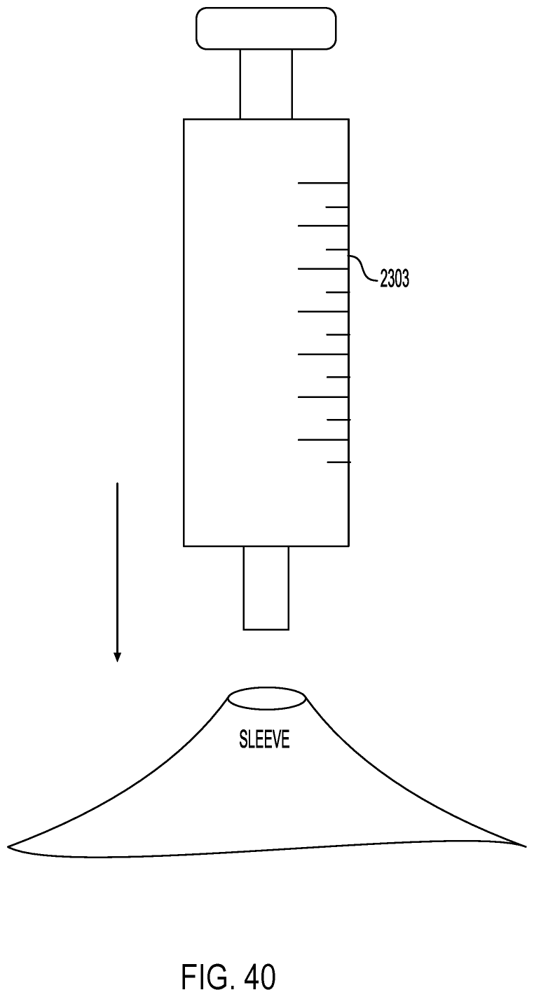

FIG. 39 illustrates methods of injection of liquid from the pack into the modular components either individually or post assembly on the affected area. In particular, FIG. 39 illustrates a dispensing pin or a static mixer pin 2301 attached to a pack containing liquid and an elastic layer 2302 at the inlet of the flexible sleeve. The pin is pierced into the rubber layer creating a system with one way flow, with the elastic layer 2302 creating a seal upon removal of the pin. As such, the elastic layer 2302 is self-healing with flow being allowed when pierced, and flow stopped when the pin 2301 is removed. In this embodiment, the pack is attached with a dispensing pin which is pushed into the sleeve, which contains a section of elastic which when pierced creates an opening for liquid to flow into the component. Upon removal of the pin, the elastic creates a seal to prevent liquid from flowing back. In one example, the dispensing pin consists of a hollow tubing with a sharp tip to pierce the elastic rubber. In another example, the dispensing pin is replaced with a mixing tip which mixes 2 or more liquids, with a sharp tip to pierce the elastic rubber. FIG. 40 illustrates a cylindrical casing containing a piston 2303 which is used to push liquid through the dispensing head. embodiment, the liquid is injected into the component through the use of a cylindrical accessory which consists of a piston to push liquid through the dispensing pin or a valve, into the sleeve.

### G. Therapeutic Accessory

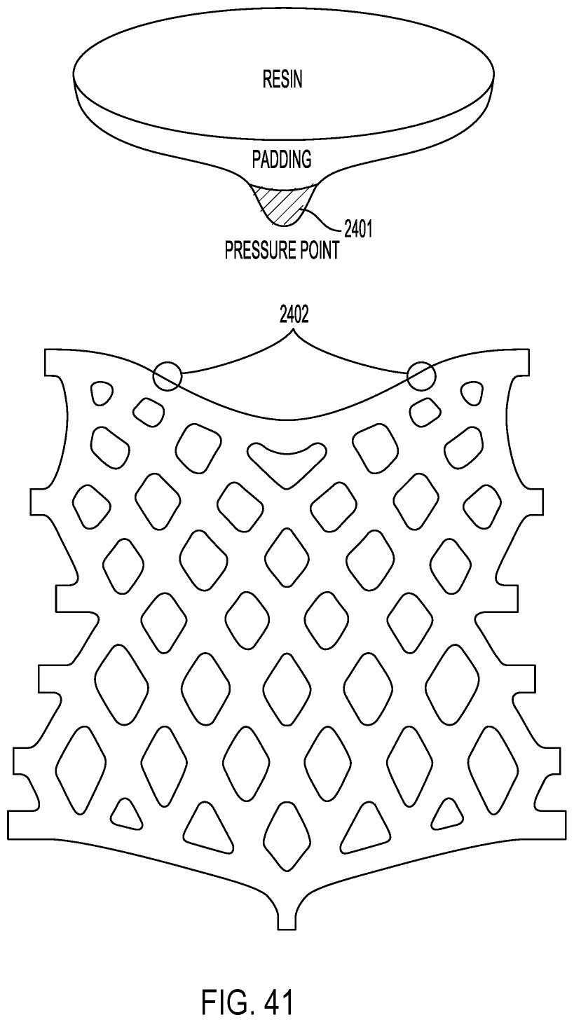

FIG. 41 illustrates a pressure point 2401 consisting of a semi-rigid flexible foam-like material, pressing against the surface of the skin. FIG. 41 further illustrates a hemi-spherical protrusion 2402 on the lateral or dorsal surface as required, to be used for therapy and recovery of the affected area. In this embodiment, the padding layer is attached with a singular spherical or hemi-spherical attachment on the inside of the layer, in contact with the skin, made of foam-like material.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

We claim:

1. A device configured to be coupled to a lattice structure, wherein the lattice structure is configured to support a first joint of a body, and wherein the lattice structure defines a plurality of apertures configured to allow for a flow of air and water to the first joint, the device comprising:
a strut having a first end and a second end opposite the first end;
a coupling mechanism coupled to the first end of the strut, wherein the coupling mechanism is configured to couple the device to the lattice structure; and
an immobilization component coupled to the strut, wherein the immobilization component is configured to support a second joint of the body to thereby at least partially immobilize the second joint.

2. The device of claim 1, wherein the immobilization component is coupled to the second end of the strut.

3. The device of any one of claims 1-2, wherein the strut is rigid.

4. The device of any one of claims 1-3, wherein a length of the strut is adjustable.

5. The device of any one of claims 1-4, wherein the immobilization component comprises a semi-circular shape.

6. The device of any one of claims 1-4, wherein the immobilization component comprises a circular shape.

7. The device of any one of claims 1-6, wherein the immobilization component includes a closed-cell foam or a silicone material configured to contact the second joint of the body.

8. The device of any one of claims 1-7, wherein the coupling mechanism is configured to be removable coupled to the lattice structure.

9. The device of claim 8, wherein the coupling mechanism comprises a flexible open ring structure configured to transition from an open position in which the coupling mechanism is positioned around a portion of the lattice structure and a closed position in which the coupling mechanism secures the device to the lattice structure.

10. The device of any one of claims 1-7, wherein the coupling mechanism is configured to be fixed to the lattice structure.

11. The device of claim 10, wherein the coupling mechanism comprises a rigid open ring structure with a fixed opening, wherein the lattice structure is configured to receive a liquid resin and a catalyst mixture that transforms into a solid such that the lattice structure hardens to thereby support the first joint of the body, wherein the rigid open ring structure is configured to be positioned around the lattice structure prior to the lattice structure receiving the liquid resin and the catalyst mixture, and wherein a diameter of the lattice structure is larger than the fixed opening after receiving the liquid resin and the catalyst mixture such that the coupling mechanism is permanently fixed to the lattice structure once the liquid resin and the catalyst mixture transforms into a solid.

12. The device of any one of claims 1-11, wherein the coupling mechanism comprises:
a first coupling mechanism configured to be coupled to a first aperture of the plurality of apertures of the lattice structure; and
a second coupling mechanism configured to be coupled to a second aperture of the plurality of apertures of the lattice structure.

13. The device of any one of claims 1-11, wherein the coupling mechanism comprises:
a first coupling mechanism configured to be coupled to a first aperture of the plurality of apertures of the lattice structure; and
a second coupling mechanism configured to be coupled to the first aperture of the plurality of apertures of the lattice structure.

14. The device of any one of claims 1-13, wherein the strut comprises a first strut, the device further comprising a second strut having a first end and a second end, wherein the coupling mechanism coupled to the first end of the second strut, and wherein the immobilization component coupled to the second strut.

15. The device of any one of claims 1-14, wherein the immobilization component comprises a first closed ring positioned adjacent the second end of the strut, and a second closed ring positioned between the first closed ring and the first end of the strut.

16. The device of any one of claims 1-15, wherein a diameter of the immobilization component is adjustable.

17. The device of any one of claims 1-16, wherein the strut includes:
a first through-hole including a first plurality of teeth; and
a second through-hole including a second plurality of teeth, wherein the first plurality of teeth and the second plurality of teeth are configured to hold the immobilization component in place.

18. A device configured to be coupled to a lattice structure, wherein the lattice structure is configured to support a first joint of a body, and wherein the lattice structure defines a plurality of apertures configured to allow for a flow of air and water to the first joint, the device comprising:
a strap having a first end and a second end opposite the first end;
a coupling mechanism coupled to the first end of the strap, wherein the coupling mechanism is configured to couple the device to the lattice structure; and
an immobilization component configured to be held in place by the strap, wherein the immobilization component is configured to support a second joint of the body to thereby at least partially immobilize the second joint.

19. The device of claim 18, wherein at least a portion of the strap includes a hook and loop fastener.

20. The device of claim 19, wherein the strap is wrapped around the immobilization component and then attached to itself to thereby secure the immobilization component to the second joint.

21. The device of any one of claims 18-20, wherein the immobilization component is rigid.

22. The device of any one of claims 18-21, wherein the second end of the strap includes a second coupling mechanism, and wherein the second coupling mechanism is configured to couple the second end of the strap to the lattice structure.

23. The device of any one of claims 18-22, wherein the coupling mechanism comprises one of a t-anchor, an s-hook, or a c-hook.

24. A device configured to be coupled to a lattice structure, wherein the lattice structure is configured to support a first joint of a body, and wherein the lattice structure defines a plurality of apertures configured to allow for a flow of air and water to the first joint, the device comprising:
a first elastic cable having a first end and a second end opposite the first end; and
a first coupling mechanism coupled to the first end of the first elastic cable, wherein the first coupling mechanism is configured to couple the first elastic cable to a first aperture of the plurality of apertures of the lattice structure,
wherein the second end of the first elastic cable and the second end of the second elastic cable are configured to hold a utensil.

25. The device of claim 24, wherein the first coupling mechanism and the second coupling mechanism comprise one of a t-anchor, an s-hook, or a c-hook.

26. The device of any one of claims 24-25, further comprising:
a second elastic cable having a first end and a second end opposite the first end;
a second coupling mechanism coupled to the first end of the second elastic cable, wherein the second coupling mechanism is configured to couple the second elastic cable to a second aperture of the plurality of apertures of the lattice structure;
a third coupling mechanism positioned at the second end of the first elastic cable; and
a fourth coupling mechanism positioned at the second end of the second elastic cable,
wherein the third coupling mechanism and the fourth coupling mechanism are configured to hold the utensil.

27. A device configured to be coupled to a lattice structure, wherein the lattice structure is configured to support a first joint of a body, and wherein the lattice structure defines a plurality of apertures configured to allow for a flow of air and water to the first joint, the device comprising:
a first elastic cable having a first end and a second end opposite the first end;
a first coupling mechanism positioned at the first end of the first elastic cable, wherein the first coupling mechanism is configured to couple the first elastic cable to an aperture of the plurality of apertures of the lattice structure;
a second coupling mechanism positioned at the second end of the first elastic cable, wherein the second coupling mechanism is configured to couple the first elastic cable to a first digit of the body;
a second elastic cable having a first end and a second end opposite the first end;
a third coupling mechanism positioned at the first end of the second elastic cable, wherein the third coupling mechanism is configured to couple the second elastic cable to an aperture of the plurality of apertures of the lattice structure; and
a fourth coupling mechanism positioned at the second end of the second elastic cable, wherein the fourth coupling mechanism is configured to couple the second elastic cable to a second digit of the body.

28. The device of claim 27, further comprising:
a third elastic cable having a first end and a second end opposite the first end:
a fifth coupling mechanism positioned at the first end of the third elastic cable, wherein the fifth coupling mechanism is configured to couple the third elastic cable to an aperture of the plurality of apertures of the lattice structure;
a sixth coupling mechanism positioned at the second end of the third elastic cable, wherein the sixth coupling mechanism is configured to couple the third elastic cable to a third digit of the body;
a fourth elastic cable having a first end and a second end opposite the first end;
a seventh coupling mechanism positioned at the first end of the fourth elastic cable, wherein the seventh coupling mechanism is configured to couple the fourth elastic cable to an aperture of the plurality of apertures of the lattice structure; and
an eighth coupling mechanism positioned at the second end of the fourth elastic cable, wherein the eighth coupling mechanism is configured to couple the fourth elastic cable to a fourth digit of the body.

29. A device configured to be coupled to a lattice structure, wherein the lattice structure is configured to support a first joint of a body, and wherein the lattice structure defines a plurality of apertures configured to allow for a flow of air and water to the first joint, the device comprising:
a through-hole positioned at a first end of the device, wherein the through-hole is configured to receive at least a portion of a thumb of the body when in use;
a utensil positioned at a second end of the device opposite the first end; and
an attachment mechanism configured to be removably positioned at least partially within an aperture of the plurality of apertures of the lattice structure.

30. The device of claim 29, wherein the attachment mechanism comprises a protrusion configured to contact an edge of the aperture to thereby prevent slipping or turning of the device when in use.

31. The device of any one of claims 29-31, wherein the utensil positioned at the second end of the device comprises one of a spoon, a fork, or a knife.

32. A system comprising:
a first lattice structure, wherein the first lattice structure is configured to support a first area of a body, and wherein the first lattice structure defines a first plurality of apertures configured to allow for a flow of air and water to the first area of the body;
a second lattice structure, wherein the second lattice structure is configured to support a second area of the body, and wherein the second lattice structure defines a second plurality of apertures configured to allow for a flow of air and water to the second area of the body; and
a coupling mechanism configured to couple the first lattice structure to the second lattice structure.

33. The system of claim 32, wherein the coupling mechanism comprises a plurality of struts each having a first end and a second end opposite the first end, wherein the first end of each of the plurality of struts are coupled to the first plurality of apertures of the first lattice structure, and wherein the second end of each of the plurality of struts are coupled to the second plurality of apertures of the second lattice structure.

34. The system of claim 32, wherein the coupling mechanism comprises a pairing mechanism.

35. The system of claim 32, wherein the coupling mechanism comprises a male protrusion and a female protrusion to thereby establish a fluidic connection between the first lattice structure and the second lattice structure.

36. The system of claim 32, wherein the coupling mechanism comprises an adhesive.

37. The system of any one of claims 32-36, further comprising:
a third lattice structure, wherein the third lattice structure is configured to support a third area of the body, and wherein the third lattice structure defines a third plurality of apertures configured to allow for a flow of air and water to the third area of the body; and
a second coupling mechanism configured to couple the second lattice structure to the third lattice structure.

38. The system of any one of claims 32-37, wherein the coupling mechanism is configured to adjust a distance between the first lattice structure and the second lattice structure.

39. The system of any one of claims 32-38, further comprising:
a semi-rigid base coupled to the first lattice structure.

40. The system of claim 39, wherein an anterior section of the semi-rigid base comprises a foam material.

41. A method of forming a support for application to a body area, the method comprising:
positioning a lattice structure around the body area;
coupling a first portion of the lattice structure to a second portion of the lattice structure via a coupling mechanism; and
injecting a liquid resin and a catalyst mixture into a lumen of the lattice structure, wherein the liquid resin and the catalyst mixture transforms into a solid in the lumen of the lattice structure such that the lattice structure hardens into the support for application to the body area.

42. The method of claim 41, wherein the coupling mechanism comprises a locking strap having a first protrusion configured to be positioned within a first aperture of the first portion of the lattice structure and a second protrusion configured to be positioned within a second aperture of the second portion of the lattice structure.

43. The method of claim 41, wherein the second portion of the lattice structure includes a through-hole, and wherein the first portion of the lattice structure includes an extension configured to be positioned through the through-hole, wherein a diameter of the extension is greater than a diameter of the through-hole after the liquid resin and the catalyst mixture transforms into a solid.

44. The method of claim 41, wherein the coupling mechanism comprises a plurality of hook and fastener straps.

45. A method of forming a support for application to a body area, the method comprising:
positioning a lattice structure around the body area;
coupling a restraining clip to a portion of the lattice structure; and
injecting a liquid resin and a catalyst mixture into a lumen of the lattice structure, wherein the liquid resin and the catalyst mixture transforms into a solid in the lumen of the lattice structure such that the lattice structure hardens into the support for application to the body area, and wherein the restraining clip prevents the liquid resin and the catalyst mixture from entering a portion of the lumen thereby creating a void in the lumen.

46. The method of claim 45, wherein the void in the lumen creates a flexible section of the lattice structure.
